(19) United States
(12) Patent Application Publication
(10) Pub. No.: US 2026/0337279 A1
(43) Pub. Date: Oct. 29, 2026

(54) ANTI-CCR8 ANTIBODIES AND USES THEREOF

(71) Applicant: FibroGen, Inc., San Francisco, CA (US)

(72) Inventors: David T. Brousseau, Mill Valley, CA (US); Gail Walkinshaw, San Francisco, CA (US); Claudia Witschi, San Francisco, CA (US)

(21) Appl. No.: 19/104,957

(22) PCT Filed: Aug. 18, 2023
(86) PCT No.: PCT/US2023/072462

§ 371 Date: Feb. 19, 2025

Related U.S. Application Data
(60) Provisional application No. 63/399,453, filed on Aug. 19, 2022.

(51) Int. Cl. C07K 16/28; A61K 39/395; A61K 45/06

(52) U.S. Cl. CPC C07K 16/2866; A61K 39/3955; A61K 45/06; C07K 2317/24

(57) ABSTRACT

Provided herein are isolated monoclonal antibody anti-CCR8 antibodies, antigen binding fragments thereof, mutants thereof, variants thereof and uses thereof for the treatment of diseases such as cancer. The mutant antibodies have a complementarity-determining region 3 modified to eliminate post-translational modification, and to improve stability and potency of the antibody. The variant antibodies have various levels of afucosylation. Also provided herein are methods of use of the anti-CCR8 antibodies, including methods of treatment of cancer. Also provided are polynucleotides encoding the heavy chain or the light chain or the antigen-binding portion thereof described herein, and vectors, especially expression vector, including the polynucleotides described herein.

### CROSS-REFERENCE TO RELATED APPLICATIONS

This application claims benefit of priority under 35 U.S.C. § 119 (e) of U.S. Provisional Application No. 63/399,483, filed Aug. 19, 2022. The disclosure of the prior application is considered part of and is herein incorporated by reference in the disclosure of this application in its entirety.

### INCORPORATION OF SEQUENCE LISTING

The material in the accompanying sequence listing is hereby incorporated by reference into this application. The accompanying sequence listing xml file, name 148640-004502_SL.xml, was created on Aug. 18, 2023 and is 39,844 bytes.

### SUMMARY OF THE INVENTION

This invention relates to isolated anti-chemokine (C-C motif) receptor 8 (CCR8) monoclonal antibodies or antigen-binding fragments thereof, nucleic acids and expression vectors encoding the antibodies, recombinant cells containing the vectors, and compositions comprising the antibodies. Methods of making the antibodies, and methods of using the antibodies to treat diseases including cancer and/or associated complications are also provided.

CCR8 is a chemokine receptor mediating cell migration under CCL1 or CCL18 gradient (Islam et al., JEM 210 (10): 1889-1898 (2013)). Recently, CCR8 was identified as a highly specific cell-surface marker of tumor infiltrating regulatory T cells (TITRs) in human cancers with considerably higher expression in Tregs residing within tumors as compared to Tregs in circulation, and no or very low expression on other T-cell population (cytotoxic T-cells or effector T-cells, respectively). Furthermore, CCR8 is predominantly expressed on high immunosuppressive Tregs that expressed FoxP3high, CD25high, TIGIT+, LAG3+ and release high IL-10 and TGF-β. Depleting CCR8+Tregs would decrease immunosuppressives cytokines and modulate a pro-tumoral microenvironment to restore anti-tumor immunity. Interestingly, in patients with breast or pancreatic cancer, high CCR8+Treg numbers correlated with more advanced stages of the disease and decreased probably of overall survival. Therefore, CCR8 is an ideal target for cancer immunotherapies to treat and potentially cure CCR8-positive cancers.

Aspects of the invention described herein relate to humanized antibodies directed against CCR8, mutants thereof and variants thereof and the use thereof for the treatment of diseases such as cancer.

In one embodiment, the invention provides an isolated monoclonal antibody or an antigen-binding fragment thereof comprising a HCVR comprising a HCVR CDR1 sequence having the amino acid sequence GFRENTNA (SEQ ID NO: 10), a HCVR CDR2 sequence having the amino acid sequence IRSKSNSYATYY (SEQ ID NO: 11), and a HCVR CDR3 sequence having the amino acid sequence TRGSEQQYYYAMDY (SEQ ID NO: 19); and, a LCVR, comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15), wherein the antibody of antigen-binding fragment thereof specifically binds CCR8. In particular embodiments therein, the antibody comprises a HCVR of SEQ ID NO:36 and a LCVR of SEQ ID NO:32. In one particular embodiment therein, the antibody is Ab001_M4 and comprises the heavy chain of SEQ ID NO:6 and the light chain of SEQ ID NO:2.

In another embodiment, the invention provides an isolated monoclonal antibody or an antigen-binding fragment thereof including: a HC comprising sequences having at least 80% identity to SEQ ID NO:6 and the antigen binding specificity thereof; and a LC, comprising sequences having at least 80% identity to SEQ ID NO:2 and the antigen binding specificity thereof.

In another embodiment, the invention provides an isolated monoclonal antibody or an antigen-binding fragment thereof comprising (a) a HCVR comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSEQNYYYAMDY (SEQ ID NO: 16); and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or (b) a HCVR comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSENNYYYAMDY (SEQ ID NO: 17); and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or (c) a HCVR comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSENQYYYAMDY (SEQ ID NO: 18); and a LCVR, comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or (d) a HCVR comprising a HCVR CDR1 sequence having the amino acid sequence GFRENTNA (SEQ ID NO: 10), a HCVR CDR2 sequence having the amino acid sequence IRSKSNSYATYY (SEQ ID NO: 11), and a HCVR CDR3 sequence having the amino acid sequence TRGSDQNYYYAMDY (SEQ ID NO: 20); and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or (e) a HCVR comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSDNQYYYAMDY (SEQ ID NO: 21); and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or (f) a HCVR comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSDQQYYYAMDY (SEQ ID NO: 22); and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15), wherein the antibody of antigen-binding fragment thereof specifically binds CCR8. In particular embodiments therein, the antibody comprises (a) a HCVR of SEQ ID NO:33 and a LCVR of SEQ ID NO:32, (b) a HCVR of SEQ ID NO:34 and a LCVR of SEQ ID NO:32, (c) a HCVR of SEQ ID NO:35 and a LCVR of SEQ ID NO:32, (d) a HCVR of SEQ ID NO:37 and a LCVR of SEQ ID NO:32, (e) a HCVR of SEQ ID NO:38 and a LCVR of SEQ ID NO:32, or (f) a HCVR of SEQ ID NO:39 and a LCVR of SEQ ID NO: 32. In one particular embodiment therein, the antibody is (a) Ab001_M1 and comprises the heavy chain of SEQ ID NO:3 and the light chain of SEQ ID NO:2, (b) Ab001_M2 and comprises the heavy chain of SEQ ID NO:4 and the light chain of SEQ ID NO:2, (c) Ab001_M3 and comprises the heavy chain of SEQ ID NO:5 and the light chain of SEQ ID NO: 2, (d) Ab001_M5 and comprises the heavy chain of SEQ ID NO:7 and the light chain of SEQ ID NO:2, (e) Ab001_M6 and comprises the heavy chain of SEQ ID NO:8 and the light chain of SEQ ID NO:2, or (f) Ab001_M7 and comprises the heavy chain of SEQ ID NO:9 and the light chain of SEQ ID NO:2.

In another embodiment, the invention provides an isolated monoclonal antibody or an antigen-binding fragment thereof including: (a) a HC comprising sequences having at least 80% identity to SEQ ID NO:3 and the antigen binding specificity thereof; and a LC, comprising sequences having at least 80% identity to SEQ ID NO:2 and the antigen binding specificity thereof; (b) a HC comprising sequences having at least 80% identity to SEQ ID NO:4 and the antigen binding specificity thereof; and a LC, comprising sequences having at least 80% identity to SEQ ID NO:2 and the antigen binding specificity thereof; (c) a HC comprising sequences having at least 80% identity to SEQ ID NO:5 and the antigen binding specificity thereof; and a LC, comprising sequences having at least 80% identity to SEQ ID NO:2 and the antigen binding specificity thereof; (d) a HC comprising sequences having at least 80% identity to SEQ ID NO:7 and the antigen binding specificity thereof; and a LC, comprising sequences having at least 80% identity to SEQ ID NO:2 and the antigen binding specificity thereof; (e) a HC comprising sequences having at least 80% identity to SEQ ID NO:8 and the antigen binding specificity thereof; and a LC, comprising sequences having at least 80% identity to SEQ ID NO:2 and the antigen binding specificity thereof; or (f) a HC comprising sequences having at least 80% identity to SEQ ID NO:9 and the antigen binding specificity thereof; and a LC, comprising sequences having at least 80% identity to SEQ ID NO:2 and the antigen binding specificity thereof, wherein the antibody or antigen-binding fragment thereof specifically binds CCR8.

In one aspect, the HCVR sequence is SEQ ID NO:33 and/or the LCVR sequence is SEQ ID NO:32, the HCVR sequence is SEQ ID NO:34 and/or the LCVR sequence is SEQ ID NO: 32, the HCVR sequence is SEQ ID NO:35 and/or the LCVR sequence is SEQ ID NO:32, the HCVR sequence is SEQ ID NO:37 and/or the LCVR sequence is SEQ ID NO:32, the HCVR sequence is SEQ ID NO:38 and/or the LCVR sequence is SEQ ID NO:32, or the HCVR sequence is SEQ ID NO:39 and/or the LCVR sequence is SEQ ID NO:32. In some aspects, the HCVR sequence is SEQ ID NO:33 and the LCVR sequence is SEQ ID NO:32, the HCVR sequence is SEQ ID NO:34 and the LCVR sequence is SEQ ID NO:32, the HCVR sequence is SEQ ID NO:35 and the LCVR sequence is SEQ ID NO:32, the HCVR sequence is SEQ ID NO: 37 and the LCVR sequence is SEQ ID NO:32, the HCVR sequence is SEQ ID NO:38 and the LCVR sequence is SEQ ID NO:32, or the HCVR sequence is SEQ ID NO:39 and the LCVR sequence is SEQ ID NO:32.

In another aspect, the invention provides an isolated monoclonal antibody including: (a) a heavy chain comprising SEQ ID NO:3 and a light chain comprising SEQ ID NO: 2; (b) a heavy chain comprising SEQ ID NO:4 and a light chain comprising SEQ ID NO:2; (c) a heavy chain comprising SEQ ID NO:5 and a light chain comprising SEQ ID NO:2; (d) a heavy chain comprising SEQ ID NO:7 and a light chain comprising SEQ ID NO:2; (e) a heavy chain comprising SEQ ID NO:8 and a light chain comprising SEQ ID NO:2; (f) a heavy chain comprising SEQ ID NO:9 and a light chain comprising SEQ ID NO:2, wherein the antibody specifically binds CCR8.

In another aspect, the antibody is a humanized antibody. In one aspect, the antigen-binding fragment thereof is a Fab, Fab′, F(ab′)₂, F_{d}, single chain Fv or scFv, disulfide linked Fv, V-NAR domain, IgNar, intrabody, IgGACH₂, minibody, F(ab′)₃, tetrabody, triabody, diabody, single-domain antibody, DVD-Ig, Fcab, mAb₂, (scFv)₂, or scFv-Fc. In other aspects, the monoclonal antibody or antigen-binding fragment thereof binds human CCR8 with a K_{d} of less than about 25 nM, 20 nM, 15 nM, 10 nM, 5 nM, 2 nM, 1 nM, 0.5 nM or 0.01 nM. In one aspect, the antibody induces antibody-dependent cell-mediated cytotoxicity (ADCC). In another aspect, the antibody has an afucosylation level of at least 50%. In various aspect, the antibody has an afucosylation level of at least 85%.

In another embodiment, the invention provides a method of treating cancer in a subject in need thereof including administering to the subject an effective amount of any one of the isolated monoclonal antibodies or antigen-binding fragments described herein, thereby treating cancer in the subject.

In another embodiment, the invention provides a method of treating cancer in a subject in need thereof comprising administering to the subject an effective amount of at least one isolated monoclonal antibody or antigen-binding fragment thereof as provided herein. In one aspect, the method comprises administering the antibody having a HCVR comprising a CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSEQQYYYAMDY (SEQ ID NO: 19); and, a LCVR, comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15), thereby treating cancer in the subject. In particular embodiments therein, the antibody comprises a HCVR of SEQ ID NO:36 and a LCVR of SEQ ID NO: 32. In one particular embodiment therein, the antibody is Ab001_M4 and comprises the heavy chain of SEQ ID NO:6 and the light chain of SEQ ID NO:2.

In one aspect, the cancer is a hematological cancer In some aspects, the hematological cancer is selected from the group consisting of acute lymphoblastic leukemia (ALL), acute myeloid leukemia (AML), chronic lymphocytic leukemia, chronic myelogenous leukemia, hairy cell leukemia, AIDS-related lymphoma, cutaneous T-cell lymphoma, Hodgkin lymphoma, Hodgkin lymphoma, mycosis fungoides, non-Hodgkin lymphoma, primary central nervous system lymphoma, Sézary syndrome, cutaneous T-Cell lymphoma, Waldenström macroglobulinemia, diffuse large B-Cell Lymphoma (DLBCL), and multiple myeloma.

In another aspect, the cancer is a solid tumor. In some aspects, the solid tumor is selected from the group consisting of breast cancer, head and neck cancer, lung cancer, melanoma, uveal melanoma, colorectal cancer, renal carcinoma, urothelial cancer, ovarian cancer, endometrial cancer, uterine cancer, liver cancer, pancreatic cancer, cholangiocarcinoma, gastric cancer, gastroesophageal cancer, esophageal cancer, cervical cancer, squamous cell carcinoma, prostate cancer, or bladder cancer. In one aspect, the method further includes administering to the patient a chemotherapeutic agent, an anti-angiogenesis agent, a growth inhibitory agent, an immune-oncology agent, a checkpoint inhibitor, and/or an anti-neoplastic composition.

In an additional embodiment, the invention provides a polynucleotide encoding the heavy chain or the light chain or the antigen-binding portion thereof of any one of the antibodies or antigen-binding fragments thereof described herein.

In one embodiment, the invention provides a vector including the polynucleotide described herein, wherein the vector is an expression vector selected from the group consisting of a mammalian expression vector, a yeast expression vector, an insect expression vector, and a bacterial expression vector.

### BRIEF DESCRIPTION OF THE DRAWINGS

FIG. 1 is a graph illustrating the ADCC potency of antibodies having various levels of afucosylation.

FIGS. 2A-2C illustrate the deamination of Ab001_Wt antibody. FIG. 2A illustrates binding assay results with CHO-hCCR8 cells. FIG. 2B illustrates ADCC reporter assay results. FIG. 2C illustrates peptide mapping results.

FIGS. 3A-3B illustrate the activity of Ab001_Wt antibody over time. FIG. 3A illustrates binding assay results over time. FIG. 3B illustrates ADCC reporter assay results over time.

FIGS. 4A-4C illustrate binding assay results CHO.hCCR8 cells for Ab001_M1-M4 as compared to Ab001_Wt. FIG. 4A illustrates binding assay results for Ab001_M1 and Ab001_M2, as compared to Ab001_Wt. FIG. 4B illustrates binding assay results for Ab001 M3 and Ab001_M4, as compared to Ab001_Wt. FIG. 4C is a table showing the EC50 calculated in FIGS. 4A and 4B.

FIGS. 5A-5C illustrate ADCC reporter assay results obtained with CHO.hCCR8 as target cells for Ab001_M1-M4 as compared to Ab001_Wt. FIG. 5A illustrates ADCC reporter assay results for Ab001_M1 and Ab001_M2, as compared to Ab001 Wt. FIG. 5B illustrates ADCC reporter assay results for Ab001 M3 and Ab001_M4, as compared to Ab001_Wt. FIG. 5C is a table showing the EC50 calculated in FIGS. 5A and 5B.

### DETAILED DESCRIPTION OF THE INVENTION

Aspects of the invention described herein relate to humanized antibodies directed against CCR8 and the use thereof for the treatment of diseases such as cancer.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

The present invention is based on the recognition of liabilities within an anti-CCR8 antibody disclosed in International Publication WO 2023/020621, in particular the antibody defined as H11-10 having a heavy chain of SEQ ID NO:1 and a light chain of SEQ ID NO:2. These liabilities included asparaginyl residues in the antigen-binding complementarity-determining region (CDR) 3 in the HCVR which were susceptible to deamidation and loss of activity over time, and fucosylation in the Fc region which inhibited antibody dependent cellular cytotoxicity (ADCC). Due to the critical nature of the CDRs for antigen binding, any modification of these asparaginyl residues in HCVR CDR3 could reduce the effectiveness of the antibody in CCR8 target engagement.

In one embodiment, the invention provides an isolated monoclonal antibody or an antigen-binding fragment thereof comprising a HCVR comprising a HCVR CDR1 sequence having the amino acid sequence GFRENTNA (SEQ ID NO: 10), a HCVR CDR2 sequence having the amino acid sequence IRSKSNSYATYY (SEQ ID NO: 11), and a HCVR CDR3 sequence having the amino acid sequence TRGSEQQYYYAMDY (SEQ ID NO: 19) and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15), wherein the antibody of antigen-binding fragment thereof specifically binds CCR8. In particular embodiments therein, the antibody comprises a HCVR of SEQ ID NO:36 and/or a LCVR of SEQ ID NO:32, particularly a HCVR of SEQ ID NO:36 and a LCVR of SEQ ID NO:32. In one particular embodiment therein, the antibody is Ab001_M4 and comprises the heavy chain of SEQ ID NO:6 and the light chain of SEQ ID NO: 2.

In another embodiment, the invention provides an isolated monoclonal antibody or an antigen-binding fragment thereof including: a HC comprising sequences having at least 80% identity to SEQ ID NO:6 and the antigen binding specificity thereof; and a LC, comprising sequences having at least 80% identity to SEQ ID NO:2 and the antigen binding specificity thereof.

In another embodiment, the invention provides an isolated monoclonal antibody or an antigen-binding fragment thereof comprising (a) a HCVR comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSEQNYYYAMDY (SEQ ID NO: 16); and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or (b) a HCVR comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSENNYYYAMDY (SEQ ID NO: 17); and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or (c) a HCVR comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSENQYYYAMDY (SEQ ID NO: 18); and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or (d) a HCVR comprising a HCVR CDR1 sequence having the amino acid sequence GFRENTNA (SEQ ID NO: 10), a HCVR CDR2 sequence having the amino acid sequence IRSKSNSYATYY (SEQ ID NO: 11), and a HCVR CDR3 sequence having the amino acid sequence TRGSDQNYYYAMDY (SEQ ID NO: 20); and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or (e) a HCVR comprising a HCVR CDR1 sequence having the amino acid sequence GFRENTNA (SEQ ID NO: 10), a HCVR CDR2 sequence having the amino acid sequence IRSKSNSYATYY (SEQ ID NO: 11), and a HCVR CDR3 sequence having the amino acid sequence TRGSDNQYYYAMDY (SEQ ID NO: 21); and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or (f) a HCVR comprising a HCVR CDR1 sequence having the amino acid sequence GFRENTNA (SEQ ID NO: 10), a HCVR CDR2 sequence having the amino acid sequence IRSKSNSYATYY (SEQ ID NO: 11), and a HCVR CDR3 sequence having the amino acid sequence TRGSDQQYYYAMDY (SEQ ID NO: 22); and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15), wherein the antibody of antigen-binding fragment thereof specifically binds CCR8. In particular embodiments therein, the antibody comprises (a) a HCVR of SEQ ID NO:33 and a LCVR of SEQ ID NO:32, (b) a HCVR of SEQ ID NO:34 and a LCVR of SEQ ID NO:32, (c) a HCVR of SEQ ID NO:35 and a LCVR of SEQ ID NO:32, (d) a HCVR of SEQ ID NO:37 and a LCVR of SEQ ID NO:32, (e) a HCVR of SEQ ID NO:38 and a LCVR of SEQ ID NO:32, or (f) a HCVR of SEQ ID NO:39 and a LCVR of SEQ ID NO: 32. In one particular embodiment therein, the antibody is (a) Ab001_M1 and comprises the heavy chain of SEQ ID NO:3 and the light chain of SEQ ID NO:2, (b) Ab001_M2 and comprises the heavy chain of SEQ ID NO:4 and the light chain of SEQ ID NO:2, (c) Ab001_M3 and comprises the heavy chain of SEQ ID NO:5 and the light chain of SEQ ID NO: 2, (d) Ab001_M5 and comprises the heavy chain of SEQ ID NO:7 and the light chain of SEQ ID NO:2, (e) Ab001_M6 and comprises the heavy chain of SEQ ID NO:8 and the light chain of SEQ ID NO:2, or (f) Ab001_M7 and comprises the heavy chain of SEQ ID NO:9 and the light chain of SEQ ID NO:2.

In another embodiment, the invention provides an isolated monoclonal antibody or an antigen-binding fragment thereof including: (a) a HC comprising sequences having at least 80% identity to SEQ ID NO:3 and the antigen binding specificity thereof; and a LC, comprising sequences having at least 80% identity to SEQ ID NO:2 and the antigen binding specificity thereof; (b) a HC comprising sequences having at least 80% identity to SEQ ID NO:4 and the antigen binding specificity thereof; and a LC, comprising sequences having at least 80% identity to SEQ ID NO:2 and the antigen binding specificity thereof; (c) a HC comprising sequences having at least 80% identity to SEQ ID NO:5 and the antigen binding specificity thereof; and a LC, comprising sequences having at least 80% identity to SEQ ID NO:2 and the antigen binding specificity thereof; (d) a HC comprising sequences having at least 80% identity to SEQ ID NO:7 and the antigen binding specificity thereof; and a LC, comprising sequences having at least 80% identity to SEQ ID NO:2 and the antigen binding specificity thereof; (e) a HC comprising sequences having at least 80% identity to SEQ ID NO:8 and the antigen binding specificity thereof; and a LC, comprising sequences having at least 80% identity to SEQ ID NO: 2 and the antigen binding specificity thereof; or (f) a HC comprising sequences having at least 80% identity to SEQ ID NO:9 and the antigen binding specificity thereof; and a LC, comprising sequences having at least 80% identity to SEQ ID NO:2 and the antigen binding specificity thereof, wherein the antibody or antigen-binding fragment thereof specifically binds CCR8.

The terms “antibodies,” “abs,” “immunoglobulins,” or “Igs”, in the broadest sense, refer to glycoproteins having the same structural characteristics (i.e., molecules that contain an antigen binding site that immunospecifically binds an antigen) and encompass various antibody structures, including natural or artificial, mono- or polyvalent antibodies including but not limited to monoclonal antibodies (including chimeric monoclonal antibodies, humanized monoclonal antibodies, and human monoclonal antibodies, particularly humanized monoclonal antibodies), polyclonal antibodies, single chain antibodies, antibody fragments, and multispecific antibodies (e.g., bispecific antibodies). While antibodies exhibit binding specificity to a specific antigen, immunoglobulins include both antibodies and other antibody-like molecules which lack antigen specificity. “Antibody” encompasses any polypeptide comprising an antigen-binding site regardless of the source, species of origin, method of production, and characteristics. The term “antibody” may also broadly refer to a molecule comprising CDR1, CDR2, and CDR3 of a heavy chain and CDR1, CDR2, and CDR3 of a light chain, wherein the molecule is capable of binding to an antigen. In some embodiments, an antibody comprises a HCVR and a LCVR. In some embodiments, an antibody comprises at least one heavy chain (HC) comprising a HCVR and at least a portion of a heavy chain constant region, and at least one light chain (LC) comprising a LCVR region and at least a portion of a light chain constant region. In some embodiments, an antibody comprises two heavy chains, wherein each heavy chain comprises a HCVR and at least a portion of a heavy chain constant region, and two light chains, wherein each light chain comprises a LCVR and at least a portion of a light chain constant region.

In some aspects, the isolated monoclonal antibody or antigen-binding fragment thereof comprises the HCVR sequence of SEQ ID NO:33 and/or the LCVR sequence of SEQ ID NO: 32, the HCVR sequence of SEQ ID NO:34 and/or the LCVR sequence of SEQ ID NO: 32, the HCVR sequence of SEQ ID NO:35 and/or the LCVR sequence of SEQ ID NO:32, the HCVR sequence of SEQ ID NO:37 and/or the LCVR sequence of SEQ ID NO:32, the HCVR sequence of SEQ ID NO: 8 and/or the LCVR sequence of SEQ ID NO:32, or the HCVR sequence is SEQ ID NO:39 and/or the LCVR sequence is SEQ ID NO:32.

In other aspects, the isolated monoclonal antibody or antigen-binding fragment thereof comprises the HCVR sequence of SEQ ID NO:33 and the LCVR sequence of SEQ ID NO: 32, the HCVR sequence of SEQ ID NO:34 and the LCVR sequence of SEQ ID NO:32, the HCVR sequence of SEQ ID NO:35 and the LCVR sequence of SEQ ID NO:32, the HCVR sequence of SEQ ID NO:37 and the LCVR sequence of SEQ ID NO:32, the HCVR sequence of SEQ ID NO:38 and the LCVR sequence of SEQ ID NO:32, or the HCVR sequence of SEQ ID NO: 39 and the LCVR sequence of SEQ ID NO:32.

“Native antibodies” and “intact immunoglobulins”, or the like, are usually heterotetrameric glycoproteins of about 150,000 daltons, composed of two identical light (L) chains and two identical heavy (H) chains. The light chains from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes). For example, IgG antibodies include, but are not limited to, IgG1 (comprising a γ1 constant region), IgG2 (comprising a γ2 constant region), IgG3 (comprising a γ3 constant region), and IgG4 (comprising a γ4 constant region) antibodies; IgA antibodies include, but are not limited to, IgAQ1 (comprising an al constant region) and IgA2 (comprising an α2 constant region) antibodies; and IgM antibodies include, but are not limited to, IgM1 (comprising an μ1 constant region) and IgM2 (comprising an μ2 constant region). The heavy-chain constant domains that correspond to the different classes of immunoglobulins are called α, δ, ε, γ, and μ, respectively. The subunit structures and three-dimensional configurations of different classes of immunoglobulins are well known.

The intact antibody may have one or more “effector functions” which refer to those biological activities attributable to the Fc region (a native sequence Fc region or amino acid sequence variant Fc region or any other modified Fc region) of an antibody. Examples of antibody effector functions include C1q binding; complement dependent cytotoxicity; Fc receptor binding; ADCC; phagocytosis; down regulation of cell surface receptors (e.g., B cell receptor (BCR); and cross-presentation of antigens by antigen presenting cells or dendritic cells.

Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains. Each variable region includes three segments called complementarity-determining regions (CDRs) or hypervariable regions and more highly conserved portions of variable domains are called the framework region (FR). The variable domains of heavy and light chains each includes four FR regions, largely adopting a β-sheet configuration, connected by three CDRs, which form loops connecting, and in some cases forming part of the β-sheet structure. The CDRs in each chain are held together in close proximity by the FRs and, with the CDRs from the other chain, contribute to the formation of the antigen-binding site of antibodies (see Kabat et al., NIH Publ. No. 91-3242, Vol. I, pages 647-669 [1991]). The constant domains are not involved directly in binding an antibody to an antigen, but exhibit various effector functions, such as participation of the antibody in antibody dependent cellular cytotoxicity.

The term “heavy chain variable region (HCVR)” as used herein refers to, at a minimum, a region comprising heavy chain CDR1 (CDR-H1), framework 2 (HFR2), CDR2 (CDR-H2), FR3 (HFR3), and CDR3 (CDR-H3). In some embodiments, a HCVR also comprises at least a portion (e.g., the whole) of an FR1 (HFR1), which is N-terminal to CDR-H1, and/or at least a portion (e.g., the whole) of an FR4 (HFR4), which is C-terminal to CDR-H3. In various embodiments, any of the HCVR sequences disclosed herein further comprise a HFR1 sequence of SEQ ID NO:23, a HFR2 sequence of SEQ ID NO:24, a HFR3 sequence of SEQ ID NO:25, and/or a HFR4 sequence of SEQ ID NO:26.

The term “heavy chain constant region” as used herein refers to a region comprising at least three heavy chain constant domains, CH1, CH2, and CH3. Non-limiting exemplary heavy chain constant regions include γ, δ, and α. Non-limiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, an antibody comprising an α constant region is an IgA antibody, an antibody comprising an ε constant region is an IgE antibody, and an antibody comprising an u constant region is an IgM antibody.

The term “light chain variable region (LCVR)” as used herein refers to a region comprising light chain CDR1 (CDR-L1), framework (FR) 2 (LFR2), CDR2 (CDR-L2), FR3 (LFR3), and CDR3 (CDR-L3). In some embodiments, a LCVR also comprises at least a portion (e.g., the whole) of an FR1 (LFR1) and/or at least a portion (e.g., the whole) of an FR4 (LFR4). In various embodiments, any of the LCVR sequences disclosed herein further comprise a LFR1 sequence of SEQ ID NO:27, a LFR2 sequence of SEQ ID NO:28, a LFR3 sequence of SEQ ID NO: 29, and/or a LFR4 sequence of SEQ ID NO:30.

The term “light chain constant region” as used herein refers to a region comprising a light chain constant domain, C L. Non-limiting exemplary light chain constant regions include λ and κ.

The term “light chain” as used herein refers to a polypeptide comprising at least a LCVR, with or without a leader sequence. In some embodiments, a light chain comprises at least a portion of a light chain constant region. The term “full-length light chain” as used herein refers to a polypeptide comprising a LCVR and a light chain constant region, with or without a leader sequence.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

In a general aspect, the invention relates to isolated monoclonal antibodies or antigen-binding fragments thereof that bind chemokine (C-C motif) receptor 8 (CCR8).

As used herein, an antibody that “specifically binds to CCR8” refers to an antibody and/or antigen binding domain that binds to CCR8, preferably human CCR8, with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. In certain embodiments, the antibody and/or antigen-binding domain binds to cynomolgus CCR8. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system.

The smaller the value of the KD of an antibody, the higher affinity that the antibody binds to a target antigen.

As used herein the term “IC50” refers to the half maximal inhibitory concentration of a monoclonal or bispecific antibody or antigen-binding fragment thereof of the invention. IC50 is a measure of the potency of the monoclonal or bispecific antibody or antigen-binding fragment thereof of the invention for inhibiting the binding of CCL1 to CCR8 or inhibiting the function of CCR8 in a cell. In certain embodiments, the monoclonal antibody or antigen-binding fragment thereof or the bispecific antibody or antigen-binding fragment thereof has a KD of less than about 10⁻⁷ M, less than about 10⁻⁸ M, less than about 10⁻⁹ M, less than about 10⁻¹⁰ M, less than about 10⁻¹¹ M, less than about 10⁻¹² M, or less than about 10⁻¹³ M.

As used herein the term “EC₅₀” refers to the half maximal effective concentration of a monoclonal or bispecific antibody or antigen-binding fragment thereof of the invention. EC₅₀ refers to the concentration of a monoclonal or bispecific antibody or antigen-binding fragment thereof for inducing a biological response (e.g., cell death) halfway between the baseline and maximum over a specified exposure time. In certain embodiments, the monoclonal antibody or antigen-binding fragment thereof or the bispecific antibody or antigen-binding fragment thereof has an EC₅₀ of less than about 1 μM, about 1000 nM to about 100 nM, about 100 nM to about 10 nM, about 10 nM to about 1 nM, about 1000 μM to about 500 μM, about 500 μM to about 200 μM, less than about 200 μM, about 200 μM to about 150 μM, about 200 μM to about 100 μM, about 100 μM to about 10 μM, or about 10 μM to about 1 μM.

In one aspect, the HCVR sequence is SEQ ID NO:33 and/or the LCVR sequence is SEQ ID NO:32, the HCVR sequence is SEQ ID NO:34 and/or the LCVR sequence is SEQ ID NO: 32, the HCVR sequence is SEQ ID NO:35 and/or the LCVR sequence is SEQ ID NO:32, the HCVR sequence is SEQ ID NO:36 and/or the LCVR sequence is SEQ ID NO:32, the HCVR sequence is SEQ ID NO:37 and/or the LCVR sequence is SEQ ID NO:32, the HCVR sequence is SEQ ID NO:38 and/or the LCVR sequence is SEQ ID NO:32, or the HCVR sequence is SEQ ID NO:39 and/or the LCVR sequence is SEQ ID NO:32.

In another aspect, the invention provides an isolated monoclonal antibody including: (a) a heavy chain comprising SEQ ID NO:3 and a light chain comprising SEQ ID NO: 2; (b) a heavy chain comprising SEQ ID NO:4 and a light chain comprising SEQ ID NO:2; (c) a heavy chain comprising SEQ ID NO:5 and a light chain comprising SEQ ID NO:2; (d) a heavy chain comprising SEQ ID NO:7 and a light chain comprising SEQ ID NO:2; (e) a heavy chain comprising SEQ ID NO:8 and a light chain comprising SEQ ID NO:2; (f) a heavy chain comprising SEQ ID NO:9 and a light chain comprising SEQ ID NO:2, wherein the antibody specifically binds CCR8.

In one aspect, the antibody is a humanized antibody. “Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a CDR of the recipient are replaced by residues from a CDR of a non-human species (donor antibody) such as mouse, rat or rabbit having the desired specificity, affinity, and capacity. In some instances, Fv FR residues of the human immunoglobulin are replaced by corresponding non-human residues. Furthermore, humanized antibodies may comprise residues which are found neither in the recipient antibody nor in the imported CDR or FR sequences. These modifications are made to further refine and maximize antibody performance. In general, the humanized antibody will comprise substantially all of at least one, and typically two, variable domains, in which all or substantially all of the CDRs correspond to those of a non-human immunoglobulin and all or substantially all of the FRs are those of a human immunoglobulin sequence. The humanized antibody optimally also will comprise at least a portion of an immunoglobulin constant region (Fc), typically that of a human immunoglobulin. For further details, see Jones et al., Nature, 321:522-525 (1986); Reichmann et al., Nature, 332:323-329 (1988); and Presta, Curr. Op. Struct. Biol., 2:593-596 (1992). The humanized antibody includes a PRIMATIZED™ antibody wherein the antigen-binding region of the antibody is derived from an antibody produced by immunizing macaque monkeys with the antigen of interest.

Methods for humanizing non-human antibodies are well known in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 [1988]), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human FR for the humanized antibody (Sims et al., J. Immunol., 151:2296 (1993); Chothia et al., J. Mol. Biol., 196:901 [1987]). Another method uses a particular FR derived from the consensus sequence of all human antibodies of a particular subgroup of light or heavy chains. The same FR may be used for several different humanized antibodies (Carter et al., Proc. Natl. Acad. Sci. USA, 89:4285 (1992); Presta et al., J. Immunol., 151:2623 [1993]).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993). Human antibodies can also be derived from phage-display libraries (Hoogenboom et al., J. Mol. Biol., 227:381 (1991); Marks et al., J. Mol. Biol., 222:581-597 [1991]).

Antibodies may be humanized by replacing sequences of the Fv variable region which are not directly involved in antigen binding with equivalent sequences from human Fv variable regions. General reviews of humanized chimeric antibodies are provided by Morrison et al., (Science 229:1202-1207 (1985)) and by Oi et al. (BioTechniques 4:214 (1986)). Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from for example, an antibody producing hybridoma. The recombinant DNA encoding the humanized or chimeric antibody, or fragment thereof, can then be cloned into an appropriate expression vector.

Humanized antibodies can alternatively be produced by CDR substitution (U.S. Pat. No. 5,225,539; Jones, Nature 321:552-525 (1986); Verhoeyan et al., Science 239:1534 (1988); and Beidler, J. Immunol. 141:4053-4060 (1988)).

In another aspect, the antigen-binding fragment thereof is a Fab, Fab′, F(ab′)₂, F_{d}, single chain Fv or scFv, disulfide linked F v, V-NAR domain, IgNar, intrabody, IgGACH₂, minibody, F(ab′)₃, tetrabody, triabody, diabody, single-domain antibody, DVD-Ig, Fcab, mAb₂, (scFv)₂, or scFv-Fc.

Experimentally, antibodies can be cleaved with the proteolytic enzyme papain, which causes each of the heavy chains to break, producing three separate antibody fragments. “Antibody fragments” include a portion of an intact antibody, preferably the antigen binding or variable region of the intact antibody. Examples of antibody fragments include Fab, Fab′ and F(ab′)2, Fc fragments or Fc-fusion products, single-chain Fvs (scFv), disulfide-linked Fvs (sdfv) and fragments including either a VL or VH domain; diabodies, tribodies and the like (Zapata et al. Protein Eng. 8(10): 1057-1062 [1995]). The term “antibody fragment” or “antigen binding portion” (of antibody) includes, but is not limited to, fragments that are capable of binding antigen. The two units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding but is important in later processes involved in ridding the body of the antigen.

The Fab fragment contains the constant domain of the light chain and the first constant domain (CH1) of the heavy chain. “Fab” refers to an antibody fragment with a molecular mass of approximately 50,000 daltons and has an activity of binding to the antigen. It comprises approximately half of the N-terminal side of the heavy chain and the whole of the light chain connected by a disulphide bridge. The Fab can be obtained in particular by treatment of immunoglobulin by a protease, papain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group.

F(ab′)2 antibody fragments originally were produced as pairs of Fab′ fragments which have hinge cysteines between them. Other chemical couplings of antibody fragments are also known. The term “F(ab′)₂” designates a fragment of approximately 100,000 daltons and an activity of binding to the antigen. This fragment is slightly larger than two Fab fragments connected via a disulphide bridge in the hinge region. These fragments are obtained by treating an immunoglobulin with a protease, pepsin. The Fab fragment can be obtained from the F(ab′)2 fragment by cleaving of the disulphide bridge of the hinge region.

The Fc region of an antibody is the tail region of an antibody that interacts with cell surface receptors and some proteins of the complement system. This property allows antibodies to activate the immune system. In IgG, IgA and IgD antibody isotypes, the Fc region is composed of two identical protein fragments, derived from the second and third constant domains of the antibody's two heavy chains; IgM and IgE Fc regions contain three heavy chain constant domains (CH domains 2-4) in each polypeptide chain. The Fc regions of IgGs bear a highly conserved N-glycosylation site. Glycosylation of the Fc fragment is essential for Fc receptor-mediated activity. The N-glycans attached to this site are predominantly core-fucosylated diantennary structures of the complex type. In addition, small amounts of these N-glycans also bear bisecting GlcNAc and α-2,6 linked sialic acid residues.

Fc-Fusion proteins (also known as Fc chimeric fusion protein, Fc-Ig, Ig-based Chimeric Fusion protein and Fc-tag protein) are composed of the Fc domain of IgG genetically linked to a peptide or protein of interest. Fc-Fusion proteins have become valuable reagents for in vivo and in vitro research. The Fc-fused binding partner can range from a single peptide, a ligand that activates upon binding with a cell surface receptor, signaling molecules, the extracellular domain of a receptor that is activated upon dimerization or as a bait protein that is used to identify binding partners in a protein microarray. One of the most valuable features of the Fc domain in vivo, is it can dramatically prolong the plasma half-life of the protein of interest, which for bio-therapeutic drugs, results in an improved therapeutic efficacy; an attribute that has made Fc-Fusion proteins attractive bio-therapeutic agents.

The Fc fusion protein may be part of a pharmaceutical composition including an Fc fusion protein and a pharmaceutically acceptable carrier excipients or carrier. Pharmaceutically acceptable carriers, excipients or stabilizers are well known in the art (Remington's Pharmaceutical Sciences, 16th edition, Osol, A. Ed. (1980)). Acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG).

“Fv” is the minimum antibody fragment which contains a complete antigen-recognition and -binding site. The dimers of “scFv” correspond to two scFv molecules connected together by a peptide bond. This Fv chain is frequently the result of the expression of a fusion gene including the genes coding for VH and VL connected by a linker sequence coding a peptide. The human scFv fragment may include CDR regions that are maintained in an appropriate conformation, preferably by means of the use of genetic recombination techniques. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

“Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the sFv to form the desired structure for antigen binding. For a review of sFv see Pluckthun in The Pharmacology of Monoclonal Antibodies, vol. 113, Rosenburg and Moore eds., Springer-Verlag, New York, pp. 269-315 (1994).

The “dsFv” fragment is a VH-VL heterodimer stabilized by a disulphide bridge; it may be divalent (dsFV₂). Fragments of divalent Sc(Fv)₂ or multivalent antibodies may form spontaneously by the association of monovalent scFvs or be produced by connecting scFvs fragments by peptide binding sequences.

The Fc fragment is the support for the biological properties of the antibody, in particular its ability to be recognized by immunity effectors or to activate the complement. It consists of constant fragments of the heavy chains beyond the hinge region.

The term “diabodies” signifies small antibody fragments having two antigen fixing sites. These fragments comprise, in the same VH-VL polypeptide chain, a variable heavy chain domain VH connected to a variable light chain domain VL. Using a binding sequence that is too short to allow the matching of two domains of the same chain, the matching with two complementary domains of another chain necessarily occurs and thus two antigen fixing sites are created.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 [1985]). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10:163-167 [1992]). According to another approach, F(ab′)2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

In other aspects, the monoclonal antibody or antigen-binding fragment thereof binds human CCR8 with a K_{d} of less than about 25 nM, 20 nM, 15 nM, 10 nM, 5 nM, 2 nM, 1 nM, 0.5 nM or 0.01 nM.

The term “antigen-binding domain” refers to the part of an antibody molecule that comprises the area specifically binding to or complementary to a part or all of an antigen. Where an antigen is large, an antibody may only bind to a particular part of the antigen. The “epitope” or “antigenic determinant” is a portion of an antigen molecule that is responsible for interactions with the antigen-binding domain of an antibody. An antigen-binding domain may be provided by one or more antibody variable domains (e.g., a so-called Fd antibody fragment consisting of a VH domain). An antigen-binding domain may comprise an antibody LCVR and an antibody HCVR.

An “antigen” according to the invention covers any substance that will elicit an immune response. In particular, an “antigen” relates to any substance, preferably a peptide or protein, that reacts specifically with antibodies or T-lymphocytes (T cells). According to the present invention, the term “antigen” comprises any molecule which comprises at least one epitope. Preferably, an antigen in the context of the present invention is a molecule which, optionally after processing, induces an immune reaction. According to the present invention, any suitable antigen may be used, which is a candidate for an immune reaction, wherein the immune reaction is preferably a cellular immune reaction. In the context of the embodiments of the present invention, the antigen is preferably presented by a cell, preferably by an antigen presenting cell which includes a diseased cell, in particular a cancer cell, in the context of MHC molecules, which results in an immune reaction against the antigen. An antigen is preferably a product which corresponds to or is derived from a naturally occurring antigen. Such naturally occurring antigens include tumor antigens.

The term “epitope” refers to an antigenic determinant in a molecule such as an antigen, i.e., to a part in or fragment of the molecule that is recognized by the immune system. An epitope of a protein such as a tumor antigen preferably comprises a continuous or discontinuous portion of said protein.

The terms “epitope”, “antigen peptide”, “antigen epitope”, “immunogenic peptide” and “MHC binding peptide” can be used interchangeably herein and preferably relate to an incomplete representation of an antigen which is preferably capable of eliciting an immune response against the antigen or a cell expressing or comprising and preferably presenting the antigen.

The term “binding-affinity” generally refers to the strength of the sum total of noncovalent interactions between a single binding site of a molecule (e.g., an antibody), and its binding partner. A variety of methods of measuring binding affinity or binding activity are known in the art, any of which can be used for purposes of the present invention. Specific illustrative embodiments are described in the following.

As used herein, “specific binding” refers to antibody binding to a predetermined antigen. Typically, the antibody binds with an affinity corresponding to a K_{D} of about 10⁻⁸ M or less and binds to the predetermined antigen with an affinity (as expressed by K_{D}) that is at least 10-fold less, and preferably at least 100-fold less than its affinity for binding to a non-specific antigen (e.g., BSA, casein) other than the predetermined antigen or a closely related antigen. Alternatively, the antibody can bind with an affinity corresponding to a K_{A} of about 10⁶ M⁻¹, or about 10⁷ M⁻¹, or about 10⁸ M⁻¹, or 10⁹ M⁻¹ or higher, and binds to the predetermined antigen with an affinity (as expressed by K_{A}) that is at least 10-fold higher, and preferably at least 100-fold higher than its affinity for binding to a non-specific antigen (e.g., BSA, casein) other than the predetermined antigen or a closely-related antigen

The term “k_{d}” (sec⁻¹), as used herein, is intended to refer to the dissociation rate constant of a particular antibody-antigen interaction. This value is also referred to as the off value. The term “K_{D}” (M⁻¹), as used herein, is intended to refer to the dissociation equilibrium constant of a particular antibody-antigen interaction.

In some embodiments, the CCR8-binding antibody of the invention has a dissociation constant (K_{d}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M) for CCR8, e.g., for human CCR8. In certain embodiments, the CCR8-binding antibody has a dissociation constant (K_{d}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M) for CCR8, e.g., for human CCR8.

The term “kₐ” (M⁻¹ sec⁻¹), as used herein, is intended to refer to the association rate constant of a particular antibody-antigen interaction. The term “K_{A}” (M), as used herein, is intended to refer to the association equilibrium constant of a particular antibody-antigen interaction.

As describes herein, the anti-CCR8 antibodies can also be referred to as a WT anti-CCR8 antibody (having a HCVR having the sequence of SEQ ID NO:31 and/or a LCVR having the sequence of SEQ ID NO:32), and as mutant thereof. As used herein, the term “mutant” antibody refers to antibodies that have mutations in the CDR3 to eliminate post-translational modification (deamidation and isomerization) of the asparaginyl residue at N104 and/or N105 in the wildtype sequence. Mutation of the 3 amino acid sequence 103-105, DNN, reduce or prevent deamidation and isomerization yielding improvements to stability and potency of the antibodies. In particular, mutation of the DNN sequence to EQQ prevents deamidation and retains binding to CCR8 equivalent to the parent antibody. This modification extends the active half life of the mutated antibody substantially over that of the parent antibody.

The antibodies having a HCVR sequence of any one of SEQ ID NO:33-39 and a LCVR having the sequence of SEQ ID NO:32 are referred to as the mutant anti-CCR8 antibodies. In particular, the antibody having the HCVR sequence of SEQ ID NO:36 and a LCVR sequence of SEQ ID NO:32 is a preferred mutant anti-CCR8 antibody.

In one aspect, the antibody induces antibody-dependent cell-mediated cytotoxicity (ADCC). ADCC is a mechanism of cell-mediated immune defense whereby an effector cell of the immune system actively lyses a target cell, whose membrane-surface antigens have been bound by specific antibodies. It is one of the mechanisms through which antibodies, as part of the humoral immune response, can act to limit and contain infection. ADCC is independent of the immune complement system that also lyses targets but does not require any other cell. ADCC requires an effector cell which classically is known to be natural killer (NK) cells that typically interact with immunoglobulin G (IgG) antibodies. However, macrophages, neutrophils and eosinophils can also mediate ADCC, such as eosinophils killing certain parasitic worms known as helminths via IgE antibodies. In general, ADCC has typically been described as the immune response to antibody-coated cells leading ultimately to the lysing of the infected or non-host cell. In recent literature, its importance in regard to treatment of cancerous cells and deeper insight into its deceptively complex pathways have been topics of increasing interest to medical researchers.

As used herein, the term “variant” antibody refers to antibodies that have variable level of fucosylation. Afucosylated monoclonal antibodies are monoclonal antibodies produced in an engineered cell or under select conditions so that the oligosaccharides in the Fc regions of the antibody have reduced or no fucose sugar units compared to production in a non-engineered cell or under normal conditions. When antibodies are afucosylated, ADCC is increased. Most approved monoclonal antibodies are of the IgG1 isotype, where two N-linked biantennary complex-type oligosaccharides are bound to the Fc region. The Fc region exercises the effector function of ADCC through its interaction with leukocyte receptors of the FcγR family. ADCC is important in the efficacy of cancer antibodies, but with many approved cancer antibodies there is less ADCC than could be desired due to nonspecific IgG competing with the drugs for binding to FcγIIIa on natural killer cells. Afucosylated monoclonal antibodies overcome this problem through improved FcγIIIa binding.

In their review, Pereira et al. (MABS 2018, VOL. 10, NO. 5, 693-711), discussed the relevance of antibody core fucosylation to ADCC, different strategies to produce afucosylated antibodies, and an update of afucosylated antibody drugs currently undergoing clinical trials as well as those that have been approved. The disclosure in the article is hereby incorporated in this application in its entirety.

In another aspect, the antibody has an afucosylation level of at least 50%. For example, the antibody has an afucosylation level of at least 50%, 55%, 60%, 65%, 70%, 75%, 80%, 85%, 90%, 95%, 96%, 97%, 98%, 99% or more.

In various aspect, the antibody has an afucosylation level of at least 85%. For example, the antibody has an afucosylation level of at least 85%, 86%, 87%, 88%, 89%, 90%, 91%, 92%, 93%, 94%, 95%, 96%, 97%, 98%, 99% or more.

In another embodiment, the invention provides a method of treating cancer in a subject in need thereof including administering to the subject an effective amount of any one of the isolated monoclonal antibodies or antigen-binding fragments described herein, thereby treating cancer in the subject.

In another embodiment, the invention provides a method of treating cancer in a subject in need thereof comprising administering to the subject an effective amount of at least one isolated monoclonal antibody or antigen-binding fragment thereof as provided herein, thereby treating cancer in the subject. In one aspect, the method comprises administering the antibody having a HCVR comprising a CDR1 sequence having the amino acid sequence GFRENTNA (SEQ ID NO:10), a HCVR CDR2 sequence having the amino acid sequence IRSKSNSYATYY (SEQ ID NO:11), and a HCVR CDR3 sequence having the amino acid sequence TRGSEQQYYYAMDY (SEQ ID NO:19) and a LCVR comprising a LCVR CDR1 sequence having the amino acid sequence QSLLHSNGNTY (SEQ ID NO:13), a LCVR CDR2 sequence having the amino acid sequence YRMSNR (SEQ ID NO:14), and a LCVR CDR3 sequence having the amino acid sequence MQHLEYPFT (SEQ ID NO:15), thereby treating cancer in the subject. In particular embodiments therein, the antibody comprises a HCVR of SEQ ID NO:36 and a LCVR of SEQ ID NO:32. In one particular embodiment therein, the antibody is Ab001_M4 and comprises the heavy chain of SEQ ID NO:6 and the light chain of SEQ ID NO:2.

The term “subject” as used herein refers to any individual or patient to which the subject methods are performed. Generally, the subject is human, although as will be appreciated by those in the art, the subject may be an animal. Thus, other animals, including vertebrate such as rodents (including mice, rats, hamsters and guinea pigs), cats, dogs, rabbits, farm animals including cows, horses, goats, sheep, pigs, chickens, etc., and primates (including monkeys, chimpanzees, orangutans and gorillas) are included within the definition of subject.

The term “treatment” is used interchangeably herein with the term “therapeutic method” and refers to both 1) therapeutic treatments or measures that cure, slow down, lessen symptoms of, and/or halt progression of a diagnosed pathologic conditions or disorder, and 2) and prophylactic/preventative measures. Those in need of treatment may include individuals already having a particular medical disorder as well as those who may ultimately acquire the disorder (i.e., those needing preventive measures). “Treatment” covers any administration or application of a therapeutic for a disease (also referred to herein as a “disorder” or a “condition”) in a mammal, including a human, and includes inhibiting the disease or progression of the disease, inhibiting or slowing the disease or its progression, arresting its development, partially or fully relieving the disease, partially or fully relieving one or more symptoms of a disease, or restoring or repairing a lost, missing, or defective function; or stimulating an inefficient process. The term “treatment” also includes reducing the severity of any phenotypic characteristic and/or reducing the incidence, degree, or likelihood of that characteristic.

The terms “therapeutically effective amount”, “effective dose,” “therapeutically effective dose”, “effective amount,” or the like refer to that amount of the subject compound that will elicit the biological or medical response of a tissue, system, animal or human that is being sought by the researcher, veterinarian, medical doctor or other clinician. Generally, the response is either amelioration of symptoms in a patient or a desired biological outcome (e.g., treatment of the cancer). A therapeutically effective amount of CCR8 antagonist of the invention may vary according to factors such as the disease state, age, sex, and weight of the individual, and the ability of the antagonist to elicit a desired response in the individual. A therapeutically effective amount encompasses an amount in which any toxic or detrimental effects of CCR8 antagonist are outweighed by the therapeutically beneficial effects.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration. In various embodiments, anti-CCR8 antibodies may be administered subcutaneously or intravenously.

The antibody and antigen binding fragments thereof described herein can be prepared in pharmaceutical compositions comprising the antibody or antigen-binding fragment thereof and a pharmaceutically acceptable carrier. A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

The subject compositions may be formulated into preparations in solid, semi-solid, liquid, or gaseous forms; including, but not limited to, tablets, capsules, powders, granules, ointments, solutions, suppositories, enemas, injections, inhalants, and aerosols.

In various embodiments, compositions comprising CCR8 antagonist are provided in formulations with a wide variety of pharmaceutically acceptable carriers (see, e.g., Gennaro, Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Nonlimiting exemplary carriers include saline, buffered saline, dextrose, water, glycerol, ethanol, and combinations thereof.

In various embodiments, compositions comprising CCR8 antagonist may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives.

In various embodiments, the compositions may be formulated for inhalation, for example, using pressurized acceptable propellants such as dichlorodifluoromethane, propane, nitrogen, and the like.

The compositions may also be formulated, in various embodiments, into sustained release microcapsules, such as with biodegradable or non-biodegradable polymers. A non-limiting exemplary biodegradable formulation includes poly lactic acid-glycolic acid (PLGA) polymer. A non-limiting exemplary non-biodegradable formulation includes a polyglycerin fatty acid ester. Certain methods of making such formulations are described, for example, in EP 1125584 A1.

Pharmaceutical dosage packs comprising one or more containers, each containing one or more doses of anti-CCR8 antibody and antigen binding fragments thereof, are also provided. In some embodiments, a unit dosage is provided wherein the unit dosage contains a predetermined amount of a composition comprising anti-CCR8 antibody and antigen binding fragments thereof, with or without one or more additional agents. In some embodiments, such a unit dosage is supplied in single-use prefilled syringe for injection. In various embodiments, the composition contained in the unit dosage may comprise saline, sucrose, or the like; a buffer, such as phosphate, or the like; and/or be formulated within a stable and effective pH range. Alternatively, in some embodiments, the composition may be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition of the invention comprises heparin and/or a proteoglycan.

Pharmaceutical compositions are administered in an amount effective for treatment or prophylaxis of the specific indication. The therapeutically effective amount is typically dependent on the weight of the subject being treated, his or her physical or health condition, the extensiveness of the condition to be treated, or the age of the subject being treated.

In some embodiments, anti-CCR8 antibody and antigen binding fragments thereof may be administered in an amount in the range of about 50 μg/kg body weight to about 50 mg/kg body weight per dose. In some embodiments, anti-CCR8 antibody and antigen binding fragments thereof may be administered in an amount in the range of about 100 μg/kg body weight to about 50 mg/kg body weight per dose. In some embodiments, anti-CCR8 antibody and antigen binding fragments thereof may be administered in an amount in the range of about 100 μg/kg body weight to about 20 mg/kg body weight per dose. In some embodiments, anti-CCR8 antibody and antigen binding fragments thereof may be administered in an amount in the range of about 0.5 mg/kg body weight to about 20 mg/kg body weight per dose.

In some embodiments, anti-CCR8 antibody and antigen binding fragments thereof may be administered in an amount in the range of about 10 mg to about 1,000 mg per dose. In some embodiments, anti-CCR8 antibody and antigen binding fragments thereof may be administered in an amount in the range of about 20 mg to about 500 mg per dose. In some embodiments, anti-CCR8 antibody and antigen binding fragments thereof may be administered in an amount in the range of about 20 mg to about 300 mg per dose. In some embodiments, anti-CCR8 antibody and antigen binding fragments thereof may be administered in an amount in the range of about 20 mg to about 200 mg per dose.

The anti-CCR8 antibody and antigen binding fragments thereof compositions may be administered as needed to subjects. In some embodiments, an effective dose of anti-CCR8 antibody and antigen binding fragments thereof is administered to a subject one or more times. In various embodiments, an effective dose of anti-CCR8 antibody and antigen binding fragments thereof is administered to the subject once a month, less than once a month, such as, for example, every two months, every three months, or every six months. In other embodiments, an effective dose of anti-CCR8 antibody and antigen binding fragments thereof is administered more than once a month, such as, for example, every two weeks, every week, twice per week, three times per week, daily, or multiple times per day. An effective dose of anti-CCR8 antibody and antigen binding fragments thereof is administered to the subject at least once. In some embodiments, the effective dose of CCR8 antagonist may be administered multiple times, including for periods of at least a month, at least six months, or at least a year. In some embodiments, anti-CCR8 antibody and antigen binding fragments thereof is administered to a subject as needed to alleviate one or more symptoms of a condition.

The antibodies described herein can be used for the treatment of cancer.

Cancer is a group of diseases involving abnormal cell growth with the potential to invade or spread to other parts of the body. In 2015, about 90.5 million people had cancer, about 14.1 million new cases occur a year and it caused about 8.8 million deaths (15.7% of deaths). The most common types of cancer in males are lung cancer, prostate cancer, colorectal cancer and stomach cancer. In females, the most common types are breast cancer, colorectal cancer, lung cancer and cervical cancer.

The term “cancer” refers to a group of diseases characterized by abnormal and uncontrolled cell proliferation starting at one site (primary site) with the potential to invade and to spread to other sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof.

As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, pancreatic, brain, and head and neck cancers, melanoma, sarcoma, multiple myeloma, leukemia, lymphoma, and the like.

In one aspect, the cancer is a hematological cancer or a solid tumor.

Cancer that begins in blood-forming tissue, such as the bone marrow, or in the cells of the immune system are referred to as hematological cancer, or blood cancer. Hematological cancers affect the production and function of blood cells, and are classified in three main types: leukemia, lymphoma, and multiple myeloma.

As used herein, “leukemia” refers to a blood cancer caused by the rapid production of abnormal white blood cells. Examples of leukemia include acute lymphoblastic leukemia (ALL), acute myeloid leukemia (AML), chronic lymphocytic leukemia, chronic myelogenous leukemia, and hairy cell leukemia.

As used herein, “lymphoma” refers to a type of blood cancer that affects the lymphatic system. Examples of lymphoma include AIDS-related lymphoma, cutaneous T-cell lymphoma, Hodgkin lymphoma, mycosis fungoides, non-Hodgkin lymphoma, primary central nervous system lymphoma, Sézary syndrome, cutaneous T-Cell lymphoma, and Waldenström macroglobulinemia.

As used herein, “myeloma” is a cancer of the plasma cells. Examples of myeloma include chronic myeloproliferative neoplasms, Langerhans cell histiocytosis, multiple myeloma, plasma cell neoplasm, myelodysplastic syndromes, and myelodysplastic/myeloproliferative neoplasms.

In some embodiments, the anti-CCR8 antibody of the invention can be used alone, or alternatively used in combination with any other suitable compound known to be able to treat the disease or indication.

In some aspects administration can be in combination with one or more additional therapeutic agents. The phrases “combination therapy”, “combined with” and the like refer to the use of more than one medication or treatment simultaneously to increase the response. The composition of the present invention might for example be used in combination with other drugs or treatment in use to treat cancer. Specifically, the administration of the composition of the present invention to a subject can be in combination with any anti-cancer therapies. Such therapies can be administered prior to, simultaneously with, or following administration of the composition of the present invention.

In certain embodiments, anti-CCR8 antibody is administered with another treatment, either simultaneously, or consecutively, to a subject, e.g., a subject having cancer. For example, anti-CCR8 antibody may be administered with one of more of: radiotherapy, surgery, or chemotherapy, e.g., targeted chemotherapy or immunotherapy. The administration of the two agents may start at times that are, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks apart, or administration of the second agent may start, e.g., 30 minutes, 60 minutes, 90 minutes, 120 minutes, 3 hours, 6 hours, 12 hours, 24 hours, 36 hours, 48 hours, 3 days, 5 days, 7 days, or one or more weeks after the first agent has been administered.

In some aspects, the hematological cancer is selected from the group consisting of acute lymphoblastic leukemia (ALL), acute myeloid leukemia (AML), chronic lymphocytic leukemia, chronic myelogenous leukemia, hairy cell leukemia, AIDS-related lymphoma, cutaneous T-cell lymphoma, Hodgkin lymphoma, mycosis fungoides, non-Hodgkin lymphoma, primary central nervous system lymphoma, Sézary syndrome, cutaneous T-Cell lymphoma, Waldenström macroglobulinemia, diffuse large B-Cell Lymphoma (DLBCL) and multiple myeloma.

Examples of solid cancer include but are not limited to, carcinoma, sarcoma, squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer.

In other aspects, the solid tumor is selected from the group consisting of breast cancer, head and neck cancer, lung cancer, melanoma, uveal melanoma, colon cancer, renal carcinoma, ovarian cancer, liver cancer, and prostate cancer.

In another embodiment, the invention provides a method of treating cancer in a subject in need thereof comprising administering to the subject an effective amount of the isolated monoclonal antibody or antigen-binding fragment thereof having a HCVR comprising a CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSEQQYYYAMDY (SEQ ID NO: 19); and, a LCVR, comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15), thereby treating cancer in the subject.

In some aspects, the methods described herein further includes administering to the patient a chemotherapeutic agent, an anti-angiogenesis agent, a growth inhibitory agent, an immune-oncology agent, and/or an anti-neoplastic composition.

A “chemotherapeutic agent” is a chemical compound that can be useful in the treatment of cancer. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem Intl. Ed. Engl, 33:183-186 (1994); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; polysaccharide complex (JHS Natural Products, Eugene, OR); razoxane; rhizoxin; sizofiran; spirogermanium; tenuazonic acid; triaziquone; 2, 2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (Bristol-Myers Squibb Oncology, Princeton, N.J.), Cremophor-free, albumin-engineered nanoparticle formulation of paclitaxel (American Pharmaceutical Partners, Schaumberg, Illinois), and doxetaxel (Rhone-Poulenc Rorer, Antony, France); chloranbucil; gerncitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib) and VEGF-A that reduce cell proliferation and pharmaceutically acceptable salts, acids or derivatives of any of the above.

Further non-limiting exemplary chemotherapeutic agents include anti-hormonal agents that act to regulate or inhibit hormone action on cancers such as anti-estrogens and selective estrogen receptor modulators (SERMs), including, for example, tamoxifen, raloxifene, droloxifene, 4-hydroxy tamoxifen, trioxifene, keoxifene, LY117018, onapristone, and toremifene; aromatase inhibitors that inhibit the enzyme aromatase, which regulates estrogen production in the adrenal glands, such as, for example, 4 (5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-alpha, Ralf and H-Ras; ribozymes such as a VEGF expression inhibitor and a HER2 expression inhibitor; vaccines such as gene therapy vaccines; rIL-2; topoisomerase 1 inhibitor; rmRH; and pharmaceutically acceptable salts, acids or derivatives of any of the above.

An “anti-angiogenesis agent” or “angiogenesis inhibitor” refers to a small molecular weight substance, a polynucleotide (including, e.g., an inhibitory RNA (RNAi or siRNA), a polypeptide, an isolated protein, a recombinant protein, an antibody, or conjugates or fusion proteins thereof, that inhibits angiogenesis, vasculogenesis, or undesirable vascular permeability, either directly or indirectly. It should be understood that the anti-angiogenesis agent includes those agents that bind and block the angiogenic activity of the angiogenic factor or its receptor. For example, an anti-angiogenesis agent is an antibody or other antagonist to an angiogenic agent, e.g., antibodies to VEGF-A (e.g., bevacizumab) or to the VEGF-A receptor (e.g., KDR receptor or Flt-1 receptor), anti-PDGFR inhibitors such as (Imatinib Mesylate), small molecules that block VEGF receptor signaling (e.g., PTK787/ZK2284, SU6668, /SUI 1248 (sunitinib malate), AMG706, or those described in, e.g., international patent application WO 2004/113304). Anti-angiogensis agents also include native angiogenesis inhibitors, e.g., angiostatin, endostatin, etc. See, e.g., Klagsbrun and D'Amore (Annu. Rev. Physiol. 1991, vol. 53:217-39); Streit and Detmar (Oncogene 2003, vol. 22:3172-3179) (e.g., Table 3 listing anti-angiogenic therapy in malignant melanoma); Ferrara & Alitalo (Nature Medicine 1999, vol 5 (12): 1359-1364); Tonini et al. (Oncogene 2003, vol. 22:6549-6556); and Sato (Int. J. Clin. Oncol. 2003, vol 8:200-206; e.g., Table 1 listing anti-angiogenic agents used in clinical trials).

A “growth inhibitory agent” as used herein refers to a compound or composition that inhibits growth of a cell (such as a cell expressing VEGF) either in vitro or in vivo. Thus, the growth inhibitory agent may be one that significantly reduces the percentage of cells (such as a cell expressing VEGF) in S phase. Examples of growth inhibitory agents include, but are not limited to, agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (Rhone-Poulenc Rorer), derived from the European yew, is a semisynthetic analogue of paclitaxel (Bristol-Myers Squibb). Paclitaxel and docetaxel promote the assembly of microtubules from tubulin dimers and stabilize microtubules by preventing depolymerization, which results in the inhibition of mitosis in cells.

The term “anti-neoplastic composition” refers to a composition useful in treating cancer comprising at least one active therapeutic agent. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents (also referred to as immuno-oncology agents), apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib platelet derived growth factor inhibitors (e.g., (Imatinib Mesylate)), a COX-2 inhibitor (e.g., celecoxib), interferons, CTLA4 inhibitors (e.g., anti-CTLA antibody ipilimumab), PD-1 inhibitors (e.g., anti-PD1 antibodies, BMS-936558), PDL1 inhibitors (e.g., anti-PDL1 antibodies, MPDL3280A), PDL2 inhibitors (e.g., anti-PDL2 antibodies), VISTA inhibitors (e.g., anti-VISTA antibodies), cytokines, antagonists (e.g., neutralizing antibodies) that bind to one or more of the following targets ErbB2, ErbB3, ErbB4, PDGFR-beta, BlyS, APRIL, BCMA, PD-1, PDL 1, PDL2, CTLA4, VISTA, or VEGF receptor(s), TRAIL/Apo2, and other bioactive and organic chemical agents, etc. Combinations thereof are also included in the invention.

“Checkpoint inhibitor therapy” is a form of cancer treatment currently that uses immune checkpoints which affect immune system functioning. Immune checkpoints can be stimulatory or inhibitory. Tumors can use these checkpoints to protect themselves from immune system attacks. Checkpoint therapy can block inhibitory checkpoints, restoring immune system function. Checkpoint proteins include programmed cell death 1 protein (PDCD1, PD-1; also known as CD279) and its ligand, PD-1 ligand 1 (PD-L1, CD274), cytotoxic T-lymphocyte-associated protein 4 (CTLA-4), A2AR (Adenosine A2A receptor), B7-H3 (or CD276), B7-H4 (or VTCN1), BTLA (B and T Lymphocyte Attenuator, or CD272), IDO (Indoleamine 2,3-dioxygenase), KIR (Killer-cell Immunoglobulin-like Receptor), LAG3 (Lymphocyte Activation Gene-3), TIM⁻³ (T-cell Immunoglobulin domain and Mucin domain 3), and VISTA (V-domain Ig suppressor of T cell activation).

Programmed cell death protein 1, also known as PD-1 and CD279 (cluster of differentiation 279), is a cell surface receptor that plays an important role in down-regulating the immune system and promoting self-tolerance by suppressing T cell inflammatory activity. PD-1 is an immune checkpoint and guards against autoimmunity through a dual mechanism of promoting apoptosis (programmed cell death) in antigen-specific T-cells in lymph nodes while simultaneously reducing apoptosis in regulatory T cells (anti-inflammatory, suppressive T cells).

PD-1 has two ligands, PD-L1 and PD-L2, which are members of the B7 family. PD-L1 protein is upregulated on macrophages and dendritic cells (DC) in response to LPS and GM-CSF treatment, and on T cells and B cells upon TCR and B cell receptor signaling, whereas in resting mice, PD-L1 mRNA can be detected in the heart, lung, thymus, spleen, and kidney. PD-L1 is expressed on almost all murine tumor cell lines, including PAI myeloma, P815 mastocytoma, and B16 melanoma upon treatment with IFN-γ. PD-L2 expression is more restricted and is expressed mainly by DCs and a few tumor lines.

CTLA4 or CTLA-4 (cytotoxic T-lymphocyte-associated protein 4), also known as CD152 (cluster of differentiation 152), is a protein receptor that, functioning as an immune checkpoint, downregulates immune responses. CTLA4 is constitutively expressed in regulatory T cells but only upregulated in conventional T cells after activation—a phenomenon which is particularly notable in cancers. CTLA4 is a member of the immunoglobulin superfamily that is expressed by activated T cells and transmits an inhibitory signal to T cells. CTLA4 is homologous to the T-cell co-stimulatory protein, CD28, and both molecules bind to CD80 and CD86, also called B7-1 and B7-2 respectively, on antigen-presenting cells. CTLA-4 binds CD80 and CD86 with greater affinity and avidity than CD28 thus enabling it to outcompete CD28 for its ligands. CTLA4 transmits an inhibitory signal to T cells, whereas CD28 transmits a stimulatory signal. CTLA4 is also found in regulatory T cells and contributes to its inhibitory function. T cell activation through the T cell receptor and CD28 leads to increased expression of CTLA-4.

There are several checkpoint inhibitors that are currently used to treat cancer. PD-1 inhibitors include Pembrolizumab (Keytruda) and Nivolumab (Opdivo). PD-L1 inhibitors include Atezolizumab (Tecentriq), Avelumab (Bavencio) and Durvalumab (Imfinzi). CTLA-4 inhibitors include Iplimumab (Yervoy). There are several other checkpoint inhibitors being developed including an anti B7-H3 antibody (MGA271), an anti-KIR antibody (Lirilumab) and an anti-LAG3 antibody (BMS-986016).

In an additional embodiment, the invention provides a polynucleotide encoding the heavy chain or the light chain or the antigen-binding portion thereof of any one of the antibodies or antigen-binding fragments thereof described herein.

The invention also provides nucleic acid molecules comprising polynucleotides that encode one or more chains of an antibody described herein, such as an anti-CCR8 antibody. In some embodiments, a nucleic acid molecule comprises a polynucleotide that encodes a heavy chain or a light chain of an antibody described herein. In some embodiments, a nucleic acid molecule comprises both a polynucleotide that encodes a heavy chain and a polynucleotide that encodes a light chain, of an antibody described herein. In some embodiments, a first nucleic acid molecule comprises a first polynucleotide that encodes a heavy chain and a second nucleic acid molecule comprises a second polynucleotide that encodes a light chain.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an antibody described herein comprises a nucleotide sequence that encodes a leader sequence, which, when translated, is located at the N-terminus of the heavy chain or light chain. As discussed above, the leader sequence may be the native heavy or light chain leader sequence or may be another heterologous leader sequence.

As used herein, the term “nucleic acid” or “oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (v) extracted from a sample. A nucleic might be employed for introduction into, i.e., transfection of, cells, in particular in the form of RNA which can be prepared by in vitro transcription from a DNA template. The RNA can moreover be modified before application by stabilizing sequences, capping, and polyadenylation.

In one embodiment, the invention provides a vector including the polynucleotide described herein, wherein the vector is an expression vector selected from the group consisting of a mammalian expression vector, a yeast expression vector, an insect expression vector, and a bacterial expression vector.

Vectors comprising polynucleotides that encode heavy chains and/or light chains of the antibodies described herein are provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004). In some embodiments, a vector is chosen for in vivo expression of CCR8 antagonist in animals, including humans. In some such embodiments, expression of the polypeptide or polypeptides is under the control of a promoter or promoters that function in a tissue-specific manner. For example, liver-specific promoters are described, e.g., in PCT Publication No. WO 2006/076288.

The term “vector”, “expression vector”, or “plasmid DNA” is used herein to refer to a recombinant nucleic acid construct that is manipulated by human intervention. A recombinant nucleic acid construct can contain two or more nucleotide sequences that are linked in a manner such that the product is not found in a cell in nature. In particular, the two or more nucleotide sequences can be operatively linked, such as a gene encoding a protein of interest, one or more protein tags, functional domains and the like.

Vectors suitable for use in preparation of proteins and/or protein conjugates include those selected from baculovirus, phage, plasmid, phagemid, cosmid, fosmid, bacterial artificial chromosome, viral DNA, Pl-based artificial chromosome, yeast plasmid, and yeast artificial chromosome. For example, the viral DNA vector can be selected from vaccinia, adenovirus, foul pox virus, pseudorabies and a derivative of SV40. One type of vector is a genomic integrated vector, or “integrated vector,” which can become integrated into the chromosomal DNA of the host cell. Another type of vector is an episomal vector, e.g., a nucleic acid capable of extra-chromosomal replication. Vectors capable of directing the expression of genes to which they are operatively linked are referred to herein as “expression vectors.” Viral vectors include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). Viral vectors are modified so the native tropism and pathogenicity of the virus has been altered or removed. The genome of a virus also can be modified to increase its infectivity and to accommodate packaging of the nucleic acid encoding the polypeptide of interest.

The nucleic acid construct of the present invention may be introduced into a cell to be altered thus allowing expression of the chimeric protein within the cell. A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as Lipofectamine™, Dojindo Hilymax™, Fugene™, jetPEI™, Effectene™ and DreamFect™.

The nucleic acid construct of the present invention may be introduced into a host cell to be altered thus allowing expression of the chimeric protein within the cell. A variety of host cells are known in the art and suitable for chimeric proteins expression. Examples of typical cell used for transfection include, but are not limited to, a bacterial cell, a eukaryotic cell, a yeast cell, an insect cell, or a plant cell. For example, E. coli, Bacillus, Streptomyces, Pichia pastoris, Salmonella typhimurium, Drosophila S2, Spodoptera SJ9, CHO, COS (e.g., COS-7), 3T3-F442A, HeLa, HUVEC, HUAEC, NIH 3T3, Jurkat, 293, 293H, or 293F.

In various embodiments, heavy chains and/or light chains of the antibodies described herein may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S and DG44 cells; PER.C6® cells (Crucell); and NSO cells. In some embodiments, heavy chains and/or light chains of the antibodies described herein may be expressed in yeast. See, e.g., U.S. Publication No. US 2006/0270045 A1. In some embodiments, a particular eukaryotic host cell is selected based on its ability to make desired post-translational modifications to the heavy chains and/or light chains of anti-CCR8 antibody. For example, in some embodiments, CHO cells produce polypeptides that have a higher level of sialylation than the same polypeptide produced in 293 cells.

In various embodiments herein, the anti-CCR8 monoclonal antibodies of the invention are expressed in cells wherein fucosylation is restricted or absent, thereby leading to an antibody with increased afucosylation. In some aspects, the antibody is selected from (a) an antibody having a heavy chain comprising SEQ ID NO:3 and a light chain comprising SEQ ID NO: 2; (b) an antibody having a heavy chain comprising SEQ ID NO:4 and a light chain comprising SEQ ID NO:2; (c) an antibody having a heavy chain comprising SEQ ID NO:5 and a light chain comprising SEQ ID NO:2; (d) an antibody having a heavy chain comprising SEQ ID NO: 6 and a light chain comprising SEQ ID NO:2; (e) an antibody having a heavy chain comprising SEQ ID NO:7 and a light chain comprising SEQ ID NO:2; (f) an antibody having a heavy chain comprising SEQ ID NO:8 and a light chain comprising SEQ ID NO:2; (h) an antibody having a heavy chain comprising SEQ ID NO:9 and a light chain comprising SEQ ID NO: 2, wherein the antibody specifically binds CCR8. In a preferred aspect, the antibody is the antibody having a heavy chain comprising SEQ ID NO:6 and a light chain comprising SEQ ID NO: 2. In these embodiments, the antibody has an afucosylation level of at least 50%. For example, the antibody has an afucosylation level of at least 50%, 55%, 60%, 65%, 70%, 75%, 80%, 85%, 90%, 95%, 96%, 97%, 98%, 99% or more.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc., Nonlimiting exemplary methods are described, e.g., in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

In some embodiments, one or more polypeptides may be produced in vivo in an animal that has been engineered or transfected with one or more nucleic acid molecules encoding the polypeptides, according to any suitable method.

Polynucleotides can be delivered to cells (e.g., a plurality of different cells or cell types including target cells or cell types and/or non-target cell types) in a vector (e.g., an expression vector). Examples of vectors include, but are not limited to, (a) non-viral vectors such as nucleic acid vectors including linear oligonucleotides and circular plasmids; artificial chromosomes such as human artificial chromosomes (HACs), yeast artificial chromosomes (YACs), and bacterial artificial chromosomes (BACs or PACs); episomal vectors; transposons (e.g., PiggyBac); and (b) viral vectors such as retroviral vectors, lentiviral vectors, adenoviral vectors, and AAV vectors. Viral vectors have several advantages for delivery of nucleic acids, including high infectivity and/or tropism for certain target cells or tissues. In some cases, a viral vector can be used to deliver the polynucleotides described herein.

### Additional Embodiments

Embodiment 1. An isolated monoclonal antibody or an antigen-binding fragment thereof comprising: a HCVR comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSEQQYYYAMDY (SEQ ID NO:19) and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15), wherein the antibody or antigen-binding fragment thereof specifically binds CCR8.

Embodiment 2. The isolated monoclonal antibody or antigen-binding fragment thereof of embodiment 1, further comprising a HFR1 sequence of SEQ ID NO:23, a HFR2 sequence of SEQ ID NO:24, a HFR3 sequence of SEQ ID NO:25, and/or a HFR4 sequence of SEQ ID NO:26.

Embodiment 3. The isolated monoclonal antibody or antigen-binding fragment thereof of embodiment 1 or 2, further comprising a LFR1 sequence of SEQ ID NO:27, a LFR2 sequence of SEQ ID NO:28, a LFR3 sequence of SEQ ID NO:29, and/or a LFR4 sequence of SEQ ID NO:30.

Embodiment 4. The isolated monoclonal antibody or antigen-binding fragment thereof of any one of embodiments 1-3, wherein the HCVR sequence is SEQ ID NO:36 and/or the LCVR sequence is SEQ ID NO:32.

Embodiment 5. The isolated monoclonal antibody or antigen-binding fragment thereof of any one of embodiments 1-4, wherein the HCVR sequence is SEQ ID NO:36 and the LCVR sequence is SEQ ID NO:32.

Embodiment 6. The isolated monoclonal antibody of any one of embodiments 1-5, wherein the heavy chain sequence is SEQ ID NO:6 and the light chain sequence is SEQ ID NO: 2.

Embodiment 7. An isolated monoclonal antibody or an antigen-binding fragment thereof comprising (a) a HCVR comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSEQNYYYAMDY (SEQ ID NO: 16) and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or (b) a HCVR comprising a HCVR CDR1 sequence having the amino acid sequence GFRENTNA (SEQ ID NO: 10), a HCVR CDR2 sequence having the amino acid sequence IRSKSNSYATYY (SEQ ID NO: 11), and a HCVR CDR3 sequence having the amino acid sequence TRGSENNYYYAMDY (SEQ ID NO: 17) and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or (c) a HCVR comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSENQYYYAMDY (SEQ ID NO: 18) and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or (d) a HCVR comprising a HCVR CDR1 sequence having the amino acid sequence GFRENTNA (SEQ ID NO: 10), a HCVR CDR2 sequence having the amino acid sequence IRSKSNSYATYY (SEQ ID NO: 11), and a HCVR CDR3 sequence having the amino acid sequence TRGSDQNYYYAMDY (SEQ ID NO: 20) and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or (e) a HCVR comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSDNQYYYAMDY (SEQ ID NO: 21) and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or (f) a HCVR comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSDQQYYYAMDY (SEQ ID NO: 22) and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15), wherein the antibody or antigen-binding fragment thereof specifically binds CCR8.

Embodiment 8. The isolated monoclonal antibody or antigen-binding fragment thereof of embodiment 7, further comprising a HFR1 sequence of SEQ ID NO:23, a HFR2 sequence of SEQ ID NO:24, a HFR3 sequence of SEQ ID NO:25, and/or a HFR4 sequence of SEQ ID NO:26.

Embodiment 9. The isolated monoclonal antibody or antigen-binding fragment thereof of embodiment 7 or 8, further comprising a LFR1 sequence of SEQ ID NO:27, a LFR2 sequence of SEQ ID NO:28, a LFR3 sequence of SEQ ID NO:28, and/or a LFR4 sequence of SEQ ID NO:29.

Embodiment 10. An isolated monoclonal antibody or an antigen-binding fragment thereof comprising (a) the HCVR sequence of SEQ ID NO:33 and the LCVR sequence of SEQ ID NO: 32; or (b) the HCVR sequence of SEQ ID NO:34 and the LCVR sequence of SEQ ID NO: 32; or (c) the HCVR sequence of SEQ ID NO:35 and the LCVR sequence of SEQ ID NO: 32; or (d) the HCVR sequence of SEQ ID NO:37 and the LCVR sequence of SEQ ID NO: 32; or (e) the HCVR sequence of SEQ ID NO:38 and the LCVR sequence of SEQ ID NO: 32; or (f) the HCVR sequence of SEQ ID NO:39 and the LCVR sequence of SEQ ID NO: 32, wherein the antibody or antigen-binding fragment thereof specifically binds CCR8.

Embodiment 11. An isolated monoclonal antibody comprising (a) the heavy chain sequence of SEQ ID NO:3 and the light chain sequence of SEQ ID NO:2; or (b) the heavy chain sequence of SEQ ID NO:4 and the light chain sequence of SEQ ID NO:2; or (c) the heavy chain sequence of SEQ ID NO:5 and the light chain sequence of SEQ ID NO:2; or (d) the heavy chain sequence of SEQ ID NO:7 and the light chain sequence of SEQ ID NO:2; or (e) the heavy chain sequence of SEQ ID NO:8 and the light chain sequence of SEQ ID NO:2; or (f) the heavy chain sequence of SEQ ID NO:9 and the light chain sequence of SEQ ID NO:2, wherein the antibody or antigen-binding fragment thereof specifically binds CCR8.

Embodiment 12. An isolated monoclonal antibody or an antigen-binding fragment thereof having at least 80% identity to an antibody of any one of embodiments 5, 6, 10, or 11, wherein the antibody or antigen-binding fragment thereof specifically binds CCR8.

Embodiment 13. The isolated monoclonal antibody or antigen-binding fragment thereof according to any one of embodiments 1-12, wherein the antibody is a humanized antibody.

Embodiment 14. The isolated monoclonal antibody or antigen-binding fragment thereof according to any one of embodiments 1-13, wherein the antigen-binding fragment thereof is an Fab, Fab′, F(ab′)2, Fd, single chain Fv or scFv, disulfide linked F v, V-NAR domain, IgNar, intrabody, IgGACH2, minibody, F(ab′)3, tetrabody, triabody, diabody, domain antibody (dAb), DVD-Ig, Fcab, mAb2, (scFv) 2, tandem scFv, DART®, TandAb, nanobody or scFv-Fc.

Embodiment 15. The isolated monoclonal antibody or antigen-binding fragment thereof of any one of embodiments 1-14, wherein said monoclonal antibody or antigen-binding fragment thereof binds human CCR8 with a Kd of less than about 25 nM, 20 nM, 15 nM, 10 nM, 5 nM, 2 nM, 1 nM, 0.5 nM or 0.01 nM.

Embodiment 16. The isolated monoclonal antibody or antigen-binding fragment thereof of any one of embodiment 1-15, which induces ADCC.

Embodiment 17. The isolated monoclonal antibody or antigen-binding fragment thereof of any one of embodiment 1-16, wherein the antibody has an afucosylation level of at least 50%.

Embodiment 18. The isolated monoclonal antibody or antigen-binding fragment thereof of any one of embodiment 1-17, wherein the antibody has an afucosylation level of at least 85%.

Embodiment 19. A method of treating cancer in a subject in need thereof comprising administering to the subject an effective amount of the isolated monoclonal antibody or antigen-binding fragment thereof of any one of embodiments 1-18, thereby treating cancer in the subject.

Embodiment 20. A method of treating cancer in a subject in need thereof comprising administering to the subject an effective amount of the isolated monoclonal antibody or antigen-binding fragment thereof of any one of embodiments 1-18, and an anti-cancer agent, thereby treating cancer in the subject.

Embodiment 21. The method of embodiment 19 or 20, wherein the cancer is a hematological cancer or a solid tumor.

Embodiment 22. The method of embodiment 21, wherein the hematological cancer is selected from the group consisting of acute lymphoblastic leukemia (ALL), acute myeloid leukemia (AML), chronic lymphocytic leukemia, chronic myelogenous leukemia, and hairy cell leukemia, AIDS-related lymphoma, cutaneous T-cell lymphoma, Hodgkin lymphoma, Hodgkin lymphoma, mycosis fungoides, non-Hodgkin lymphoma, primary central nervous system lymphoma, Sézary syndrome, cutaneous T-Cell lymphoma, Waldenström macroglobulinemia, diffuse large B-Cell Lymphoma (DLBCL), and multiple myeloma.

Embodiment 23. The method of embodiment 21, wherein the solid tumor is breast cancer, head and neck cancer, lung cancer, melanoma, uveal melanoma, colon cancer, renal carcinoma, ovarian cancer, liver cancer, or prostate cancer.

Embodiment 24. The method of embodiment 19, further comprising administering to the patient an anti-cancer agent.

Embodiment 25. The method of embodiment 20 or 24, wherein the anti-cancer agent is a chemotherapeutic agent, an anti-angiogenesis agent, a growth inhibitory agent, an immune-oncology agent, a checkpoint inhibitor and/or an anti-neoplastic composition.

Embodiment 26. A method of treating breast cancer, head and neck cancer, lung cancer, melanoma, uveal melanoma, colon cancer, renal carcinoma, ovarian cancer, liver cancer and/or prostate cancer in a subject in need thereof comprising administering to the subject an effective amount of the isolated monoclonal antibody or antigen-binding fragment thereof of any one of embodiments 1-18, thereby treating cancer in the subject.

Embodiment 27. A method of treating breast cancer, head and neck cancer, lung cancer, melanoma, uveal melanoma, colon cancer, renal carcinoma, ovarian cancer, liver cancer and/or prostate cancer in a subject in need thereof comprising administering to the subject an effective amount of the isolated monoclonal antibody or antigen-binding fragment thereof of any one of embodiments 1-18, and an anti-cancer agent, thereby treating cancer in the subject.

Embodiment 28. The method of embodiment 27, wherein the anti-cancer agent is a chemotherapeutic agent, an anti-angiogenesis agent, a growth inhibitory agent, an immune-oncology agent, a checkpoint inhibitor and/or an anti-neoplastic composition.

Embodiment 29. A method of treating cancer in a subject in need thereof comprising administering to the subject an effective amount of an isolated monoclonal antibody or antigen-binding fragment thereof of any one of embodiments 1-6, thereby treating cancer in the subject.

Embodiment 30. A method of treating breast cancer, head and neck cancer, lung cancer, melanoma, uveal melanoma, colon cancer, renal carcinoma, ovarian cancer, liver cancer and/or prostate cancer in a subject in need thereof comprising administering to the subject an effective amount of an isolated monoclonal antibody or antigen-binding fragment thereof of any one of embodiments 1-6, thereby treating cancer in the subject.

Embodiment 31. A method of treating cancer in a subject in need thereof comprising administering to the subject an effective amount of an isolated monoclonal antibody or antigen-binding fragment thereof of any one of embodiments 1-6 and an anti-cancer agent, thereby treating cancer in the subject.

Embodiment 32. A method of treating breast cancer, head and neck cancer, lung cancer, melanoma, uveal melanoma, colon cancer, renal carcinoma, ovarian cancer, liver cancer and/or prostate cancer in a subject in need thereof comprising administering to the subject an effective amount of an isolated monoclonal antibody or antigen-binding fragment thereof of any one of embodiments 1-6 and an anti-cancer agent, thereby treating cancer in the subject.

Embodiment 33. The method of embodiment 29 or 31, wherein the cancer is a hematological cancer or a solid tumor.

Embodiment 34. The method of embodiment 33, wherein the hematological cancer is selected from the group consisting of acute lymphoblastic leukemia (ALL), acute myeloid leukemia (AML), chronic lymphocytic leukemia, chronic myelogenous leukemia, and hairy cell leukemia, AIDS-related lymphoma, cutaneous T-cell lymphoma, Hodgkin lymphoma, Hodgkin lymphoma, mycosis fungoides, non-Hodgkin lymphoma, primary central nervous system lymphoma, Sézary syndrome, cutaneous T-Cell lymphoma, Waldenström macroglobulinemia, diffuse large B-Cell Lymphoma (DLBCL), and multiple myeloma.

Embodiment 35. The method of embodiment 33, wherein the solid tumor is breast cancer, head and neck cancer, lung cancer, melanoma, uveal melanoma, colon cancer, renal carcinoma, ovarian cancer, liver cancer, or prostate cancer.

Embodiment 36. The method of embodiment 29 or 30, further comprising administering to the patient an anti-cancer agent.

Embodiment 37. The method of embodiment 36, wherein the anti-cancer agent is a chemotherapeutic agent, an anti-angiogenesis agent, a growth inhibitory agent, an immune-oncology agent, a checkpoint inhibitor and/or an anti-neoplastic composition.

Embodiment 38. A polynucleotide encoding the heavy chain or the light chain or the antigen-binding portion thereof of any one of embodiments 1-18.

Embodiment 39. A vector comprising the polynucleotide of embodiment 38, wherein the vector is an expression vector selected from the group consisting of a mammalian expression vector, a yeast expression vector, an insect expression vector, and a bacterial expression vector.

Embodiment 40. A method of producing a monoclonal antibody of embodiments 6 or 11 wherein the antibody is afucosylated at least 50%, 60%, 70%, 80%, 85%, 90%, 95%, or 99%, the method comprising expressing a polynucleotide expressing the antibody in a cell engineered to reduce or prevent fucosylation of the antibody.

Presented below are examples discussing isolated anti-CCR8 antibodies as described herein. The following examples are provided to further illustrate the embodiments of the present invention but are not intended to limit the scope of the invention. While they are typical of those that might be used, other procedures, methodologies, or techniques known to those skilled in the art may alternatively be used.

### EXAMPLE

### Example 1

### Humanized Anti-CCR8 Antibodies Afucosylation

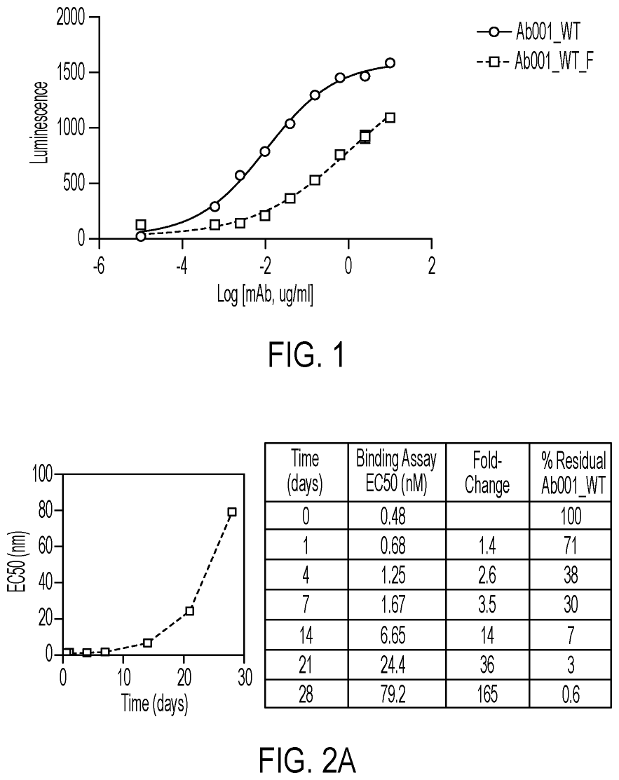

To assess the effect of the level of afucosylation on the properties of the Ab001_WT antibody, the ATUM's proprietary hypofucosylation technology (miFuc™) was applied during cell line generation to increase the level of afucosylation to >90%. A second comparator cell line with identical sequence, but without the miFuc™ hypofucosylation technology, yielded approximately 11% afucosylation. Potency of the antibodies by ADCC was evaluated using Jurkat-CD16A-NFAT with HuT78 Cells reporter assay. An approximate 100-fold increase in ADCC activity was observed with the hypofucosylated cell line over the non-hypofucosylated cell line (see Table 1 and FIG. 1).

TABLE 1
ADCC Reporter Assay EC50 Results with Hut78 Target Cells

| | EC50 | EC50 |
|---|---|---|
| Sample | (ng/ml) | (pM) |
| Ab001_WT | 10.6 | 71 |
| Ab001_WT_F | 1153 | 7687 |

### Example 2

### CCR8 Binding Assay

Antibodies were tested for binding to CCR8 using CHO-K1 Human Chemokine CCR8 Receptor Cell Line (CHO.hCCR8; Perkin Elmer Corp., Waltham MA) as follows. Approximately 5×10⁴ CHO.hCCR8 cells were resuspended in 50 μL serially diluted antibody in cold MACS buffer (Miltenyi Biotec, Inc., San Jose CA) at antibody concentrations of 10, 3.3, 1.1, 0.37, 0.12, 0.04, and 0.01 nM in wells of a V-bottom 96-well plate. After 30 min incubation on ice, 150 μL cold MACS buffer was added to each well, plates were centrifuged at 500×g for 5 min at 4 °C, and supernatants were discarded. Cells were washed twice in 200 μL cold MACS buffer and then resuspended in 50 μL anti-human IgG-Alexa Fluor 488 (The Jackson Laboratory, Bar Harbor ME) diluted 1:100 in MACS buffer. After incubation for 30 min on ice, 150 μL cold MACS buffer was added to each well, plates were centrifuged at 500×g for 5 min at 4 °C, and supernatants were discarded. Cells were washed twice in 200 μL cold MACS buffer and then resuspended in 80 μL cold MACS buffer with propidium iodide (PI, 1:1000 dilution, 1 μg/ml; ThermoFisher Scientific, Waltham MA). Antibody binding (luminescence) was measured using a CellStream flow cytometry system (Cytek Biosciences, Fremont CA).

### Example 3

### Antibody-Dependent Cell Cytotoxicity (ADCC) Assay

CHO.hCCR8 cells were seeded at a density of 1×10⁴ cells/well in 100 μL Ham's F-12 media, 10% FBS, 0.4 mg/mL G418 in white, clear bottomed, 96 well plates. After 24 hours, media was removed and 40 μL of Assay Media (One-Step Luciferase Assay System; BPS Bioscience, San Diego CA) was added. Antibody was diluted 1:10 and then serially diluted 1:4 in Assay Media, and 10 μL of each was distributed to wells and incubated for 1 hour. ADCC Bioassay Effector Cell V variant (High Affinity)—Jurkat Recombinant Cell Line (BPS Bioscience) cells were added to each well at 5×10⁴ cells/well in 50 μL aliquots. Plates were incubated for 5 hours at 37 °C, 5% CO₂ before adding 100 μL luciferase assay working solution (Luciferase Reagent Substrate diluted 1:100 in Luciferase Reagent Buffer; BPS Bioscience) and gently rocking plates for 20 minutes at room temperature. Luminescence was then measured using a luminometer.

### Example 4

### Deamidation of Ab001_Wt Antibody

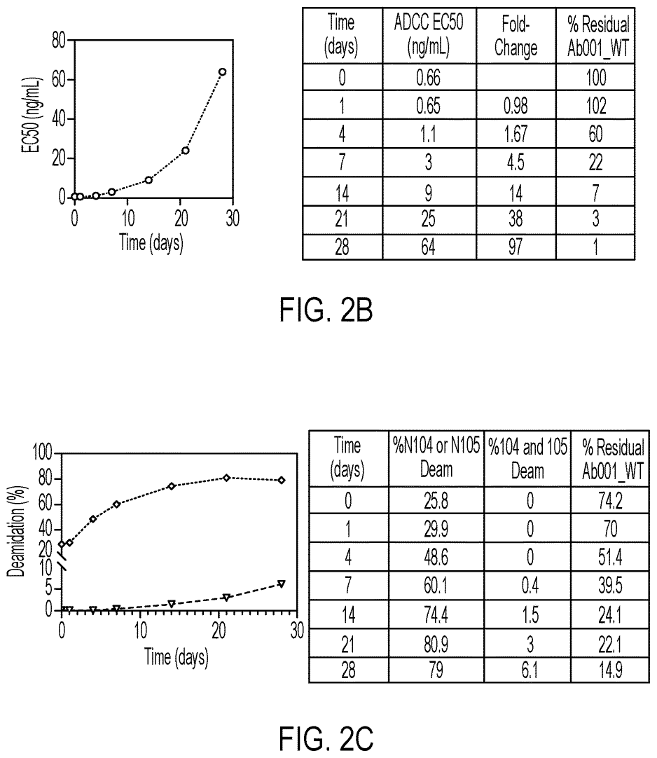

Ab001_Wt was incubated in PBS, pH 7.4 at 37 °C, and samples were collected at 0, 1, 4, 7, 14, 21, and 28 days. Samples were assayed for human CCR8 binding EC₅₀, ADCC EC₅₀, and percent deamidation at N104 and/or N105 at each time point. As illustrated in FIG. 2A and FIG. 2B, hCCR8 binding and ADCC activity decreased over time. As illustrated in FIG. 2C, the decreases seen track with deamidation of the Ab001_Wt antibody over the same time period. This demonstrates a liability in Ab001_Wt that reduces the antibody's effectiveness over time, and indicates that frequent dosing of the antibody would be required to maintain active levels of Ab001_Wt.

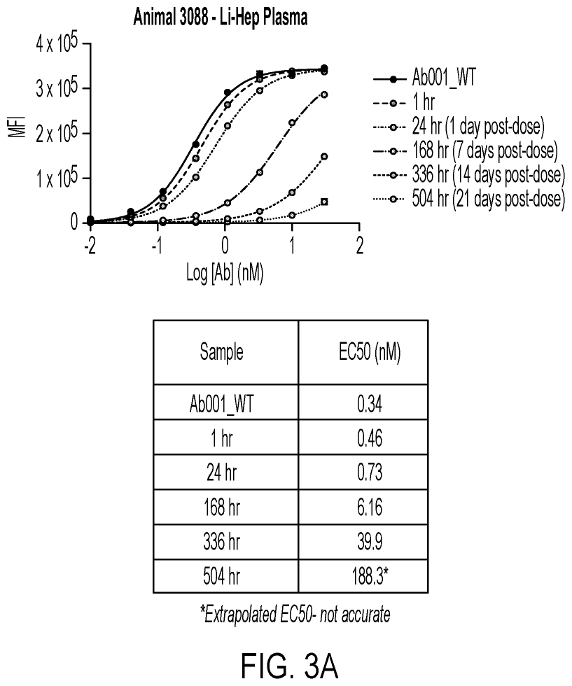

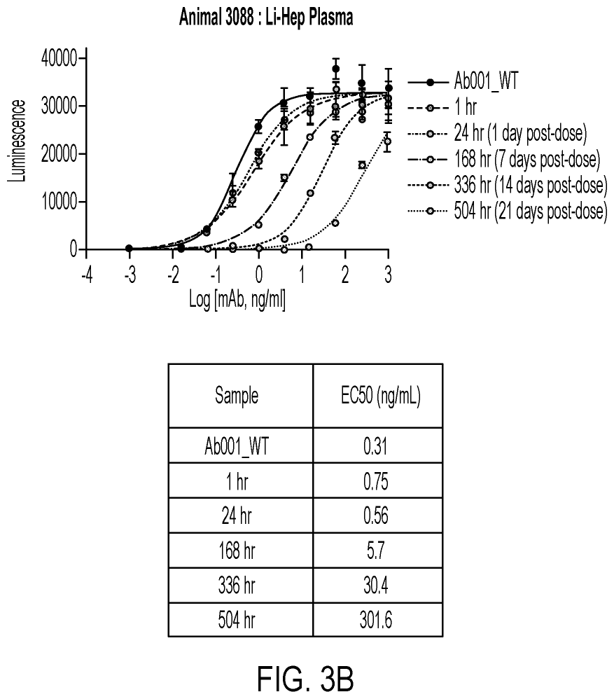

In a second experiment, plasma samples from a monkey pharmacokinetic (PK) study demonstrated a precipitous drop in both CCR8 binding (FIG. 3A) and ADCC activity (FIG. 3B) over time. This demonstrates the same liability of Ab001_Wt in animals.

### Example 5

### Construction and Testing of Mutant Antibodies

Ab001_Wt was modified in HCVR CDR3 to generate a series of antibodies with reduced susceptibility for deamidation. These antibodies modify the HCVR CDR3 sequence from amino acid 103 to 105 from DNN in the parent antibody Ab001_Wt to EQN in Ab001_M1, ENN in Ab001_M2, ENQ in Ab001_M3, EQQ in Ab001_M4, DQN in Ab001_M5, DNQ in Ab001_M6, and DQQ in Ab001_M7. Mutant antibodies were tested for CCR8 binding affinity and ADCC activity relative to the parent antibody Ab001_Wt.

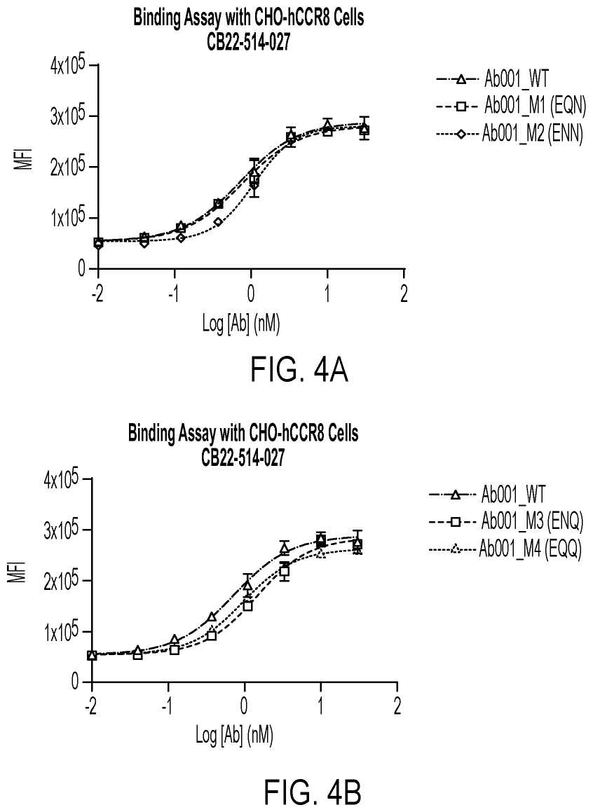

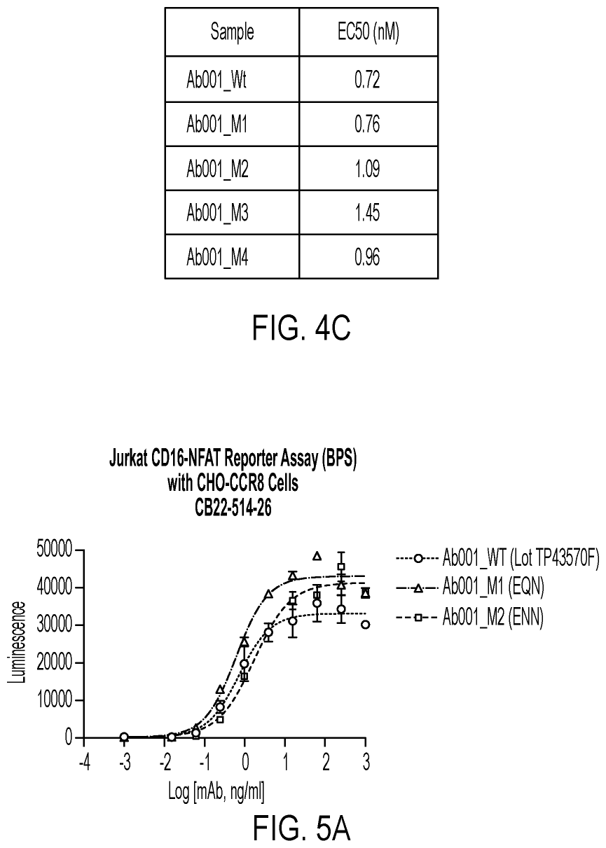

As illustrated in FIGS. 4A and 4B, the CCR8 binding of Ab001-M1, M2, M3 and M4 was measured and compared to the binding activity of Ab001_Wt. The EC50 are summarized in FIG. 4C. While modification of the CDR3 region showed some effect on binding EC₅₀, surprisingly complete replacement of the asparaginyl residues N104 and N105 in Ab001_M4 did not produce a significant change. Ab001_M4 therefore removes liability of deamidation over time and retains CCR8 binding activity.

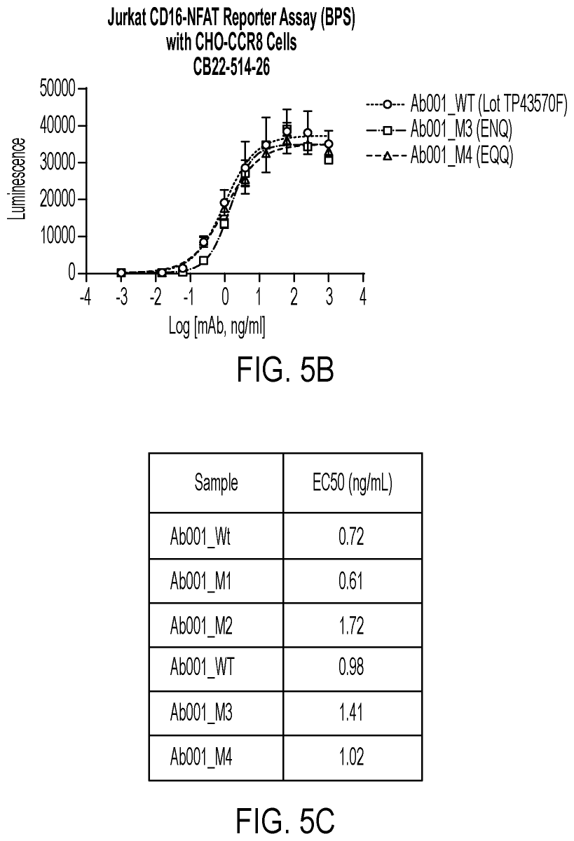

As illustrated in FIGS. 5A and 5B, the ADCC activity of Ab001-M1, M2, M3 and M4 was measured and compared to the ADCC activity of Ab001_Wt. The EC50 are summarized in FIG. 5C. As seen with CCR8 binding, while modification of the CDR3 region showed some effect on ADCC EC₅₀, surprisingly complete replacement of the asparaginyl residues N104 and N105 in Ab001_M4 did not produce a significant change. Ab001_M4 therefore removes liability of deamidation over time and retains ADCC activity.

Although the invention has been described with reference to the above examples, it will be understood that modifications and variations are encompassed within the spirit and scope of the invention. Accordingly, the invention is limited only by the following claims.

1. An isolated monoclonal antibody or an antigen-binding fragment thereof comprising:
(a) a heavy chain variable region (HCVR) comprising a HCVR complementarity-determining region (CDR1) sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSEQQYYYAMDY (SEQ ID NO: 19); and a light chain variable region (LCVR) comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15);
(b) a heavy chain variable region (HCVR) comprising a HCVR CDR 1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSEQNYYYAMDY (SEQ ID NO: 16); and a light chain variable region (LCVR) comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or
(c) a heavy chain variable region (HCVR) comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSENNYYYAMDY (SEQ ID NO: 17); and a light chain variable region (LCVR) comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or
(d) a heavy chain variable region (HCVR) comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSENQYYYAMDY (SEQ ID NO: 18); and a light chain variable region (LCVR) comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or
(e) a heavy chain variable region (HCVR) comprising a HCVR CDR1 sequence having the amino acid sequence GFRENTNA (SEQ ID NO: 10), a HCVR CDR2 sequence having the amino acid sequence IRSKSNSYATYY (SEQ ID NO: 11), and a HCVR CDR3 sequence having the amino acid sequence TRGSDQNYYYAMDY (SEQ ID NO: 20); and a light chain variable region (LCVR) comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or
(f) a heavy chain variable region (HCVR) comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSDNQYYYAMDY (SEQ ID NO: 21); and a light chain variable region (LCVR) comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15); or
(g) a heavy chain variable region (HCVR) comprising a HCVR CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSDQQYYYAMDY (SEQ ID NO: 22); and a light chain variable region (LCVR) comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15),
wherein the antibody of antigen-binding fragment thereof specifically binds CCR8.

2. The isolated monoclonal antibody or an antigen-binding fragment thereof of claim 1, wherein the monoclonal antibody or antigen-binding fragment thereof comprises:
(a) a HC comprising sequences having at least 80% identity to SEQ ID NO:6 and the antigen binding specificity thereof, and a LC comprising sequences having at least 80% identity to SEQ ID NO:2 and the antigen binding specificity thereof;
(b) a heavy chain (HC) comprising sequences having at least 80% identity to SEQ ID NO:3 and the antigen binding specificity thereof; and a light chain (LC) comprising sequences having at least 80% identity to SEQ ID NO:2 and the antigen binding specificity thereof;
(c) a HC comprising sequences having at least 80% identity to SEQ ID NO:4 and the antigen binding specificity thereof; and a LC comprising sequences having at least 80% identity to SEQ ID NO:2 and the antigen binding specificity thereof;
(d) a HC comprising sequences having at least 80% identity to SEQ ID NO:5 and the antigen binding specificity thereof; and a LC comprising sequences having at least 80% identity to SEQ ID NO:2 and the antigen binding specificity thereof;
(e) a HC comprising sequences having at least 80% identity to SEQ ID NO:7 and the antigen binding specificity thereof; and a LC comprising sequences having at least 80% identity to SEQ ID NO:2 and the antigen binding specificity thereof;
(f) a HC comprising sequences having at least 80% identity to SEQ ID NO:8 and the antigen binding specificity thereof; and a LC comprising sequences having at least 80% identity to SEQ ID NO:2 and the antigen binding specificity thereof; or
(g) a HC comprising sequences having at least 80% identity to SEQ ID NO:9 and the antigen binding specificity thereof; and a LC comprising sequences having at least 80% identity to SEQ ID NO:2 and the antigen binding specificity thereof.

3. The isolated monoclonal antibody or antigen-binding fragment thereof of claim 2, wherein:
(a) the heavy chain variable region (HCVR) sequence is SEQ ID NO: 36 and/or the light chain variable region (LCVR) sequence is SEQ ID NO: 32;
(b) the HCVR sequence is SEQ ID NO: 33 and/or the LCVR sequence is SEQ ID NO: 32,
(c) the HCVR sequence is SEQ ID NO: 34 and/or the LCVR sequence is SEQ ID NO: 32,
(d) the HCVR sequence is SEQ ID NO: 35 and/or the LCVR sequence is SEQ ID NO: 32,
(e) the HCVR sequence is SEQ ID NO: 37 and/or the LCVR sequence is SEQ ID NO: 32,
(f) the HCVR sequence is SEQ ID NO: 38 and/or the LCVR sequence is SEQ ID NO: 32,
(g) the HCVR sequence is SEQ ID NO: 39 and/or the LCVR sequence is SEQ ID NO: 32.

4. (canceled)

5. The isolated monoclonal antibody or antigen-binding fragment thereof of claim 2, wherein:
(a) the HC sequence is SEQ ID NO: 6 and the LC sequence is SEQ ID NO: 2;
(b) the HC sequence is SEQ ID NO: 3 and the LC sequence is SEQ ID NO: 2,
(c) the HC sequence is SEQ ID NO: 4 and the LC sequence is SEQ ID NO: 2,
(d) the HC sequence is SEQ ID NO: 5 and the LC sequence is SEQ ID NO: 2,
(e) the HC sequence is SEQ ID NO: 7 and the LC sequence is SEQ ID NO: 2,
(f) the HC sequence is SEQ ID NO: 8 and the LC sequence is SEQ ID NO: 2, or
(g) the HC sequence is SEQ ID NO: 9 and the LC sequence is SEQ ID NO: 2.

6-10. (canceled)

11. The isolated monoclonal antibody or antigen-binding fragment thereof according to claim 1, wherein the antibody is a humanized antibody.

12. The isolated monoclonal antibody or antigen-binding fragment thereof according to claim 1, wherein the antigen-binding fragment thereof is an Fab, Fab′, F(ab′)₂, F a, single chain Fv or scFv, disulfide linked F v, V-NAR domain, IgNar, intrabody, IgGACH₂, minibody, F(ab′)₃, tetrabody, triabody, diabody, single-domain antibody, DVD-Ig, Fcab, mAb₂, (scFv)₂, or scFv-Fc.

13. The isolated monoclonal antibody or antigen-binding fragment thereof of claim 1, wherein said monoclonal antibody or antigen-binding fragment thereof binds human CCR8 with a K a of less than about 25 nM, 20 nM, 15 nM, 10 nM, 5 nM, 2 nM, 1 nM, 0.5 nM or 0.01 nM.

14. The isolated monoclonal antibody or antigen-binding fragment thereof of claim 1, which induces antibody-dependent cell-mediated cytotoxicity (ADCC).

15. The isolated monoclonal antibody or antigen-binding fragment thereof of claim 1, wherein the antibody has an afucosylation level of at least 50%.

16. (canceled)

17. A method of treating cancer in a subject in need thereof comprising administering to the subject an effective amount of the isolated monoclonal antibody or antigen-binding fragment thereof of claim 1, thereby treating cancer in the subject.

18. The method of claim 17, wherein the cancer is a hematological cancer or a solid tumor.

19. The method of claim 18, wherein:
(a) the hematological cancer is selected from the group consisting of acute lymphoblastic leukemia (ALL), acute myeloid leukemia (AML), chronic lymphocytic leukemia, chronic myelogenous leukemia, hairy cell leukemia, AIDS-related lymphoma, cutaneous T-cell lymphoma, Hodgkin lymphoma, Hodgkin lymphoma, mycosis fungoides, non-Hodgkin lymphoma, primary central nervous system lymphoma, Sézary syndrome, cutaneous T-Cell lymphoma, Waldenström macroglobulinemia, diffuse large B-Cell Lymphoma (DLBCL), and multiple myeloma; or
(b) the solid tumor is selected from the group consisting of breast cancer, head and neck cancer, lung cancer, melanoma, uveal melanoma, colon cancer, renal carcinoma, ovarian cancer, liver cancer, and prostate cancer.

20. (canceled)

21. The method of claim 17, further comprising administering to the patient a chemotherapeutic agent, an anti-angiogenesis agent, a growth inhibitory agent, an immune-oncology agent, a checkpoint inhibitor and/or an anti-neoplastic composition.

22. A method of treating cancer in a subject in need thereof comprising administering to the subject an effective amount of the isolated monoclonal antibody or antigen-binding fragment thereof having a HCVR comprising a CDR1 sequence having the amino acid sequence GFRFNTNA (SEQ ID NO: 10), a HCVR CDR2 sequence having the amino acid sequence IRSKSNSYATYY (SEQ ID NO: 11), and a HCVR CDR3 sequence having the amino acid sequence TRGSEQQYYYAMDY (SEQ ID NO: 19); and a LCVR comprising a LCVR CDR1 sequence having the amino acid sequence QSLLHSNGNTY (SEQ ID NO: 13), a LCVR CDR2 sequence having the amino acid sequence YRMSNR (SEQ ID NO: 14), and a LCVR CDR3 sequence having the amino acid sequence MQHLEYPFT (SEQ ID NO: 15), thereby treating cancer in the subject.

23. The method of claim 22, wherein the cancer is a solid tumor.

24. The method of claim 23, wherein the solid tumor is selected from the group consisting of breast cancer, head and neck cancer, lung cancer, melanoma, uveal melanoma, colorectal cancer, renal carcinoma, urothelial cancer, ovarian cancer, endometrial cancer, uterine cancer, liver cancer, pancreatic cancer, cholangiocarcinoma, gastric cancer, gastroesophageal cancer, esophageal cancer, cervical cancer, squamous cell carcinoma, prostate cancer, or bladder cancer.

25. The method of claim 22, further comprising administering to the patient a chemotherapeutic agent, an anti-angiogenesis agent, a growth inhibitory agent, an immune-oncology agent, a checkpoint inhibitor and/or an anti-neoplastic composition.

26. A polynucleotide encoding the heavy chain or the light chain or the antigen-binding portion thereof of claim 1.

27. A vector comprising the polynucleotide of claim 26, wherein the vector is an expression vector selected from the group consisting of a mammalian expression vector, a yeast expression vector, an insect expression vector, and a bacterial expression vector.


SEQUENCE LISTING (39 sequences)

SEQ ID NO: 1 - protein, 472 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSDNNYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 239 aa, synthetic construct
METDTLLLWVLLLWVPGSTGDIVMTQSPLSLPVTPGEPASISCRSSQSLLHSNGNTYLYWFLQKPGQSPQLLIYRMSNRASGVPDRFSGSGSGTDFTLKISRVEAEDVGVYYCMQHLEYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 472 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSEQNYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 472 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSENNYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 472 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSENQYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 472 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSEQQYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 472 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSDQNYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 472 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSDNQYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 472 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSDQQYYYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 8 aa, synthetic construct
GFRFNTNA

SEQ ID NO: 11 - protein, 12 aa, synthetic construct
IRSKSNSYATYY

SEQ ID NO: 12 - protein, 14 aa, synthetic construct
TRGSDNNYYYAMDY

SEQ ID NO: 13 - protein, 11 aa, synthetic construct
QSLLHSNGNTY

SEQ ID NO: 14 - protein, 6 aa, synthetic construct
YRMSNR

SEQ ID NO: 15 - protein, 9 aa, synthetic construct
MQHLEYPFT

SEQ ID NO: 16 - protein, 14 aa, synthetic construct
TRGSEQNYYYAMDY

SEQ ID NO: 17 - protein, 14 aa, synthetic construct
TRGSENNYYYAMDY

SEQ ID NO: 18 - protein, 14 aa, synthetic construct
TRGSENQYYYAMDY

SEQ ID NO: 19 - protein, 14 aa, synthetic construct
TRGSEQQYYYAMDY

SEQ ID NO: 20 - protein, 14 aa, synthetic construct
TRGSDQNYYYAMDY

SEQ ID NO: 21 - protein, 14 aa, synthetic construct
TRGSDNQYYYAMDY

SEQ ID NO: 22 - protein, 14 aa, synthetic construct
TRGSDQQYYYAMDY

SEQ ID NO: 23 - protein, 25 aa, synthetic construct
EVQLVESGGGLVQPGGSLKLSCAAS

SEQ ID NO: 24 - protein, 17 aa, synthetic construct
MNWVRQASGKDLEWVAR

SEQ ID NO: 25 - protein, 36 aa, synthetic construct
AASVKGRFTISRDDSKNTLYLQMNSLKTEDTAVYYC

SEQ ID NO: 26 - protein, 11 aa, synthetic construct
WGQGTTVTVSS

SEQ ID NO: 27 - protein, 26 aa, synthetic construct
DIVMTQSPLSLPVTPGEPASISCRSS

SEQ ID NO: 28 - protein, 16 aa, synthetic construct
LYWFLQKPGQSPQLLI

SEQ ID NO: 29 - protein, 34 aa, synthetic construct
ASGVPDRFSGSGSGTDFTLKISRVEAEDVGVYYC

SEQ ID NO: 30 - protein, 10 aa, synthetic construct
FGQGTKLEIK

SEQ ID NO: 31 - protein, 142 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSDNNYYYAMDYWGQGTTVTVSS

SEQ ID NO: 32 - protein, 132 aa, synthetic construct
METDTLLLWVLLLWVPGSTGDIVMTQSPLSLPVTPGEPASISCRSSQSLLHSNGNTYLYWFLQKPGQSPQLLIYRMSNRASGVPDRFSGSGSGTDFTLKISRVEAEDVGVYYCMQHLEYPFTFGQGTKLEIK

SEQ ID NO: 33 - protein, 142 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSEQNYYYAMDYWGQGTTVTVSS

SEQ ID NO: 34 - protein, 142 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSENNYYYAMDYWGQGTTVTVSS

SEQ ID NO: 35 - protein, 142 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSENQYYYAMDYWGQGTTVTVSS

SEQ ID NO: 36 - protein, 142 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSEQQYYYAMDYWGQGTTVTVSS

SEQ ID NO: 37 - protein, 142 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSDQNYYYAMDYWGQGTTVTVSS

SEQ ID NO: 38 - protein, 142 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSDNQYYYAMDYWGQGTTVTVSS

SEQ ID NO: 39 - protein, 142 aa, synthetic construct
MGWSCIILFLVATATGVHSEVQLVESGGGLVQPGGSLKLSCAASGFRFNTNAMNWVRQASGKDLEWVARIRSKSNSYATYYAASVKGRFTISRDDSKNTLYLQMNSLKTEDTAVYYCTRGSDQQYYYAMDYWGQGTTVTVSS